(19) United States
(12) Patent Application Publication
(10) Pub. No.: US 2024/0420333 A1
(43) Pub. Date: Dec. 19, 2024

(54) AUTOMATED MICROBLEED SEGMENTATION USING TRANSFER LEARNING

(71) Applicant: UNIVERSITÉ LAVAL, Québec (CA)

(72) Inventors: Mahsa DADAR, Montréal (CA); Simon DUCHESNE, Québec (CA)

(21) Appl. No.: 18/702,607

(22) PCT Filed: Oct. 18, 2022
(86) PCT No.: PCT/CA2022/051532

§ 371 Date: Apr. 18, 2024

Related U.S. Application Data
(60) Provisional application No. 63/373,049, filed on Aug. 20, 2022.
(60) Provisional application No. 63/257,536, filed on Oct. 19, 2021.

(51) Int. Cl. G06T 7/00; A61B 5/00; A61B 5/02; A61B 5/055; G06T 7/11; G06V 10/32; G06V 10/764; G06V 10/774; G06V 10/82

(52) U.S. Cl. CPC G06T 7/0016; A61B 5/0042; A61B 5/02007; A61B 5/055; G06T 7/11; G06V 10/32; G06V 10/764; G06V 10/774; G06V 10/82; G06T 2207/10088; G06T 2207/20081; G06T 2207/20084; G06T 2207/30016

(57) ABSTRACT

A classifier is trained to recognize microbleed voxels within a brain image. False positives in identified microbleeds voxels can be reduced by removing identified microbleed voxels based on a comparison between an image characteristic of said identified microbleed voxels and of one or more surrounding non-microbleed voxels.

This patent application claims priority to U.S. provisional patent applications 63/257,536 filed Oct. 19, 2021 and 63/373,049 filed Aug. 20, 2022.

### TECHNICAL FIELD

This patent application relates to microbleeds, cerebrovascular disease, magnetic resonance imaging, deep neural networks and transfer learning.

### BACKGROUND

Cerebral microbleeds are small perivascular hemorrhages that can occur in both gray and white matter brain regions. They are defined as small perivascular deposits filled from hemosiderin leaking from the vessels (Greenberg et al., 2009). They are recognized as a marker of cerebral small vessel disease, along with white matter hyperintensities (WMHs) and lacunar infarcts (Wardlaw et al., 2013). Cerebral microbleeds are commonly present in patients with ischemic stroke and dementia, and are more prevalent with increasing age (Roob et al., 1999; Sveinbjornsdottir et al., 2008; Vernooij et al., 2008). The presence of microbleeds has been linked to cognitive impairment and an increased risk of dementia (Greenberg et al., 2009; Werring et al., 2004).

In vivo, cerebral microbleeds can be detected as small, round, and well-demarcated hypointense areas on susceptibility-weighted images (SWI) and T2* magnetic resonance images (MRIs) (Shams et al., 2015). Susceptibility images are hard to harmonize across scanners, and therefore a technique that can work with other contrasts would be advantageous as it would be more robust and easily disseminated to clinical centers.

Microbleeds can be identified and manually segmented by expert radiologists and neurologists, different studies use various size cut-off points to classify microbleeds, with maximum diameters ranging from 5-10 mm, and in some cases, a minimum diameter of 2 mm (Cordonnier et al., 2007). These are well correlated to histopathological findings of hemosiderin (Shoamanesh et al., 2011). In practice, microbleeds are labeled on MRI as being either “definite” or “possible” using visual rating (Gregoire et al., 2009). However, visual detection is time consuming and subject to inter and intra rater variability, particularly for smaller microbleeds, frequently overlooked by less experienced raters. Manual segmentation is laborious, time-consuming, and subject to inter and intra rater variability. There is a need therefore for reliable and practical automated microbleed segmentation tools able to produce sensitive and specific segmentations at the lesion level.

Most of the microbleed segmentation tools that are currently available are semi-automated, i.e. they require expert intervention in varying degrees to produce the final segmentation (Barnes et al., 2011; Bian et al., 2013; Fazlollahi et al., 2015; Kuijf et al., 2013, 2012; Morrison et al., 2018). Automated techniques so far have shown high accuracy at the patch level, at the expense of a high number of false positives lesions. These automated microbleed segmentation pipelines are typically based on SWI scans, which in general have a higher sensitivity and resolution for microbleed detection (Dou et al., 2016; Hong et al., 2020; Roy et al., 2015; Shams et al., 2015; Van Den Heuvel et al., 2016; Wang et al., 2017; Zhang et al., 2018). These were shown to have sensitivity (ranging from 93 to 99%) and specificity (ranging from 92 to 99%) at the patch level, but less so at the lesion level (Dou et al., 2016).

Convolutional neural networks (CNNs) in particular have been successfully employed in many image segmentation tasks. Very deep networks such as ResNet (He et al., 2016), GoogLeNet (Szegedy et al., 2015), AlexNet (Krizhevsky et al., 2017), and VGGNet (Simonyan and Zisserman, 2014) have shown impressive performances in image recognitions tasks. ResNet50 has recently been used to detect microbleeds on SWI scans, achieving an accuracy of 97.46% at the patch level, outperforming other state-of-the-art methods (Hong et al., 2020).

As mentioned, most of these deep learning based studies only report patch-level results, without assessing their techniques on a full-brain scan. The reported specificities are generally between 92-99% (Hong et al., 2020, 2019; Lu et al., 2017; Wang et al., 2017; Zhang et al., 2018). While high accuracy at a patch-level is important, at a whole brain level, even a specificity of 99% might translate into thousands of false positive voxels. In fact, applying different microbleed segmentation methods at a voxel-level, Dou et al. report precision values ranging between 5-22%, in some cases leading to 280-2500 false positives on average (Dou et al., 2016). The proposed method by Dou et al. had a much better performance in terms of precision and false positives, with a precision rate of 44.31% and an average false positive rate of 2.74, however, their reported sensitivity was relatively lower (93.16%).

Thus, improving performance at the lesion level would be desirable. Further, given that SWI are not always collected in either clinical or research settings and/or are hard to harmonize in multi-centric settings, it would be useful if a more versatile algorithm was proposed, able to segment microbleeds from other, more general MRI contrasts (e.g. T2*). To our knowledge there is no publicly available automated microbleed segmentation tool for this type of acquisition. The main challenge in developing automated microbleed segmentation tools using machine learning and in particular deep learning methods pertains to a general lack of reliable, manually labeled data.

A subcategory of MRI-identifiable cerebral microbleeds consists in those related to amyloid deposition. Those share a similar pattern to that observed in cerebral amyloid angiopathy (Nakata et al.). The Alzheimer's Association Research Rountable Workgroup further characterized these lesions and coined the phrase ARIA (amyloid-related imaging abnormalities) (Sperling et al.). Specifically, ARIA-H refers to areas of hypointensity on gradient echo MRI that are believed to represent deposits of iron in the form of hemosiderin. It has been shown in animal models that anti-amyloid β treatment removes vascular amyloid with a corresponding compromise of the integrity of the vascular wall and leakage of blood resulting in microhaemorrhages and hemosiderin deposition (Zago et al.). ARIA-H have been observed in the context of human clinical trials (Arrighi et al., Ketter et al., Vandevrede et al.). A technique able to segment microbleeds should therefore be immediately transferable to the identification, segmentation, tracking and measurement of ARIA-H.

### SUMMARY

Applicant developed an automated, more precise microbleeds segmentation tool able to use standard MRI contrasts. This tool can be used as an automated microbleed segmentation tool for this acquisition, can be trained using transfer learning to deal with a relatively small number of manually labeled microbleeds in a training dataset and can employ a post-processing to winnow out false positives. In light of promising results from other authors, applicant employed a pre-trained ResNet50 network, further tailoring it for another, relevant MRI segmentation task (i.e. classification of cerebrospinal fluid versus brain tissue) for which applicant can generate a large number of training samples. The pre-trained weights may then be used as initial weights for a microbleed segmentation network, allowing for a faster convergence with a smaller training sample. Transfer learning is a powerful paradigm that can make our algorithm potentially versatile on a number of MRI contrasts for microbleeds detection. It would be straightforward for example to use our algorithm as trained and extend it to other contrasts, such as SWI.

The tool may first be trained a ResNet50 network on another MRI segmentation task (cerebrospinal fluid versus background segmentation) using T1-weighted, T2-weighted, and T2* MRI. It can then be used for transfer learning to train the network for the detection of microbleeds with the same contrasts. As a final step, applicant employed a combination of morphological operators and rules at the local lesion level to remove false positives. In one embodiment, manual segmentations of microbleeds from 78 participants can be used to train and validate the system. Applicant assessed the impact of patch size, freezing weights of the initial layers, mini-batch size, learning rate, as well as data augmentation on the performance of the ResNet50 network. The proposed method achieved a high performance, with a patch-level sensitivity, specificity, and accuracy of 99.57%, 99.16%, and 99.93%, respectively. At a per lesion level, sensitivity, precision, and Dice similarity index values were 89.1%, 20.1%, and 0.28 for cortical GM, 100%, 100%, and 1 for deep GM, and 91.1%, 44.3%, and 0.58 for WM, respectively. The proposed microbleed segmentation method might be suitable for detecting microbleeds with high sensitivity and can be suitable for use as an automated microbleed tool.

In this specification, the term “voxel” that is normally associated with a 3D image is understood to include a pixel in the case of a 2D image.

In some embodiments, there is provided a method of processing a brain image to determine presence of microbleeds, the method comprising providing a classifier trained to recognize microbleed voxels, receiving a brain image of a subject, identifying microbleed voxels in the brain image using said classifier, and reducing false positives in the identified microbleeds voxels by removing identified microbleed voxels based on a comparison between an image characteristic of the identified microbleed voxels and of one or more surrounding non-microbleed voxels. The image characteristic may be an intensity value.

In some embodiments, there is provided a method of processing a brain image to determine the presence of microbleeds, the method comprising training a classifier on a relevant image characteristic using a first training set of brain images, further training said classifier to distinguish microbleed voxels from other brain tissue voxels using a second set of microbleed brain images segmented by one or more experts, receiving a brain image of a subject, and identifying microbleed voxels in said brain image using said classifier. The relevant image characteristic may be for distinguishing cerebral spinal fluid from gray matter/white matter.

It will also be appreciated that embodiments can be applied to a method of treating a patient using amyloid-modifying therapy, the method comprising obtaining a brain image of the patient, processing the brain image of the patient using an embodiment of the method set out above, and administering the therapy to a patient based on said identifying microbleed voxels related to amyloid-related microbleeds. Steps (a) through (c) may be repeated over time and the modifying comprises comparing changes in identified microbleed voxels over time.

### BRIEF DESCRIPTION OF THE DRAWINGS

The invention will be better understood by way of the following detailed description of embodiments of the invention with reference to the appended drawings, in which:

FIG. 1 shows a table summarizing the scanner information and acquisition parameters of the harmonized Canadian Dementia Imaging protocol for the subjects included in this study.

FIG. 2A shows examples of CSF and background patches generated for the CSF segmentation task.

FIG. 2B shows a zoom-in from FIG. 2A to better see the examples of T2* background patches generated for the CSF segmentation task.

FIG. 2C shows a zoom-in from FIG. 2A to better see the examples of T2w background patches generated for the CSF segmentation task.

FIG. 2D shows a zoom-in from FIG. 2A to better see the examples of T1w background patches generated for the CSF segmentation task.

FIG. 2E shows a zoom-in from FIG. 2A to better see the examples of T2* CSF patches generated for the CSF segmentation task.

FIG. 2F shows a zoom-in from FIG. 2A to better see the examples of T2w CSF patches generated for the CSF segmentation task.

FIG. 2G shows a zoom-in from FIG. 2A to better see the examples of T1w CSF patches generated for the CSF segmentation task.

FIG. 3A shows examples of microbleed and background patches generated for the microbleed segmentation task.

FIG. 3B shows a zoom-in from FIG. 3A to better see the examples of T2* background patches generated for the microbleed segmentation task.

FIG. 3C shows a zoom-in from FIG. 3A to better see the examples of T2w background patches generated for the microbleed segmentation task.

FIG. 3D shows a zoom-in from FIG. 3A to better see the examples of T1w background patches generated for the microbleed segmentation task.

FIG. 3E shows a zoom-in from FIG. 3A to better see the examples of T2* CSF patches generated for the microbleed segmentation task.

FIG. 3F shows a zoom-in from FIG. 3A to better see the examples of T2w CSF patches generated for the microbleed segmentation task.

FIG. 3G shows a zoom-in from FIG. 3A to better see the examples of T1w CSF patches generated for the microbleed segmentation task.

FIG. 4 shows images combination of T2*, T2 et T1 patches for generating a RGB patch for training ResNet50.

FIG. 5A shows axial slices showing FreeSurfer based tissue categories, segmented microbleeds and their dilated surrounding areas.

FIG. 5B shows a zoom-in of one example of FIG. 5A.

FIG. 5C shows axial slices showing FreeSurfer based tissue categories, segmented microbleeds and their dilated surrounding areas (zoomed-in from FIG. 5A).

FIG. 5D shows axial slices showing FreeSurfer based tissue categories, segmented microbleeds and their dilated surrounding areas (zoomed-in from FIG. 5A).

FIG. 5E shows axial slices showing FreeSurfer based tissue categories, segmented microbleeds and their dilated surrounding areas (zoomed-in from FIG. 5A).

FIG. 5F shows axial slices showing FreeSurfer based tissue categories, segmented microbleeds and their dilated surrounding areas (zoomed-in from FIG. 5A).

FIG. 6 shows a flow diagram of the microbleed segmentation pipeline.

FIG. 7 shows a microbleed distribution for each tissue type, based on the manual segmentations.

FIG. 8A shows axial slices comparing ResNet50 and BISON CSF segmentations.

FIG. 8B shows axial slice comparing ResNet50 and BISON CSF segmentations

FIG. 9 shows performance accuracy as a function of patch size, freezing of initial layers, mini-batch size, and learning rate. Colors indicate patch-level accuracy values, with warmer colors reflecting higher accuracy.

FIG. 10 shows the impact of data augmentation on performance accuracy, sensitivity and specificity at patch level. RR=Random Rotation.

FIG. 11 shows sensitivity, precision, and similarity index values for different post-processing threshold values in validation and test sets.

FIG. 12A shows axial slices comparing automated and manual segmentations.

FIG. 12B shows one enlarged axial slice comparing automated and manual segmentations from FIG. 12A.

FIG. 12C shows axial slices comparing automated and manual segmentations from FIG. 12A.

FIG. 12D shows axial slices comparing automated and manual segmentations from FIG. 12A.

FIG. 12E shows axial slices comparing automated and manual segmentations from FIG. 12A.

FIG. 12F shows axial slices comparing automated and manual segmentations from FIG. 12A.

FIG. 13A shows axial slices showing FreeSurfer based tissue categories, segmented microbleeds and their dilated surrounding areas.

FIG. 13B shows a zoom-in of the ARIA-H of the left slice of FIG. 13A.

FIG. 13C shows a zoom-in of the microbleeds of the right slice of FIG. 13A.

FIG. 14 shows the performance of U-Net models.

### DETAILED DESCRIPTION

The following is a detailed description of embodiments of the disclosure depicted in the accompanying drawings. The embodiments are in such detail as to clearly communicate the disclosure without limiting the anticipated variations of the possible embodiments and may encompass all modifications, equivalents, combinations and alternatives falling within the spirit and scope of the present disclosure. It will be appreciated by those skilled in the art that well-known methods, procedures, physical processes and components may not have been described in detail in the following so as not to obscure the specific details of the disclosed invention.

Someone versed in the art will understand that the described steps, methodologies, tools, values, etc. are only one of the many possible embodiments of the applicant's tool and that most of those presented elements (steps, methodologies, tools, etc.) can be replaced by alternative equivalents.

The present disclosure presents a method of data analysis (e.g. segmentation) enabling automated identification or confirmation manual identification of regions of interest. In an embodiment, the data can be a scan (e.g. MRI) or imagery data that can comprise one or more sets of images or volumetric images from at least a part of a patient's brain. The method can identify one more pixels or voxel of the data set that may be associated with a region of interest that can be a part or region of the patient's brain that can most likely be injured or unhealthy, such as microbleeds.

In an embodiment, applicant utilized the proposed method the developed an automated, more precise microbleeds segmentation tool able to use standard MRI contrasts. In an embodiment, the proposed tool can be used as an automated microbleed segmentation tool for this acquisition. In an embodiment, the proposed tool can be trained using transfer learning to deal with a relatively small number of manually labeled microbleeds in a training dataset. In an embodiment, the proposed tool can employ a post-processing to winnow out false positives. In light of promising results from other authors, applicant employed a pre-trained ResNet50 network, further tailoring it for another, relevant MRI segmentation task (i.e. classification of cerebrospinal fluid versus brain tissue) for which applicant can generate a large number of training samples. The pre-trained weights may then be used as initial weights for a microbleed segmentation network, allowing for a faster convergence with a smaller training sample. Transfer learning is a powerful paradigm that can make our algorithm potentially versatile on a number of MRI contrasts for microbleeds detection. It would be straightforward for example to use our algorithm as trained and extend it to other contrasts, such as SWI.

In some embodiments, there is provided a method of processing a brain image that can be used to determine presence of microbleeds. The method may comprise providing a classifier that can be trained to recognize microbleed voxels, receiving a brain image of a subject, identifying microbleed voxels in the brain image using said classifier, and reducing false positives in the identified microbleeds voxels by removing identified microbleed voxels which can be based on a comparison between an image characteristic of the identified microbleed voxels and/or of one or more surrounding non-microbleed voxels. In an embodiment, the image characteristic may be an intensity value.

Some embodiments of the method may be further comprising training a classifier on a relevant image characteristic, which can be done using a first training set of brain images. The training may be done by using the classifier to distinguish microbleed voxels from other brain tissue voxels, which can be done by using a second set of microbleed brain images segmented by one or more experts. In an embodiment, the relevant image characteristic may be for distinguishing cerebral spinal fluid from gray matter/white matter.

It will also be appreciated that embodiments can be applied to a method of treating a patient using amyloid-modifying therapy. Some embodiments of this method may be comprising obtaining a brain image of the patient, processing the brain image of the patient using an embodiment of the method as described herein, and administering the therapy to a patient based on said identifying microbleed voxels related to amyloid-related microbleeds. These steps or variations of these steps may be repeated over time and may be adjusted based on updated identified microbleed voxels over time (e.g. by comparing changes over time).

The following includes a non-limiting exemplary embodiments of the proposed method and apparatus (tool) used on real patients (i.e. participants of a study) to better understand the details and to better appreciated the qualities of the proposed method.

### MATERIALS AND METHODS

### Participants

In this exemplary study, data included 78 subjects (32 women, mean age=77.16±6.06 years) were selected from the COMPASS-ND cohort (Chertkow et al., 2019) of the Canadian Consortium on Neurodegeneration in Aging (CCNA; www.ccna-conv.ca). The CCNA is a Canadian research hub for examining neurodegenerative diseases that affect cognition in aging. Clinical diagnosis may be determined by the participating clinicians based on longitudinal clinical, screening, and MRI findings (i.e. diagnosis reappraisal may be performed using information from recruitment assessment, screening visit, clinical visit with physician input, and MRI). For details on clinical group ascertainment, see Pieruccini-Faria et al. 2021 (Pieruccini-Faria et al., 2021).

All COMPASS-ND images were read by a board-certified radiologist. Out of the whole cohort, participants can be selected based on the presence of WMHs on the fluid attenuated inversion recovery (FLAIR), as another indicator of cerebrovascular pathology, and microbleeds on T2* images. In an embodiment of an exemplary study, the sample comprised six individuals with subjective cognitive impairment (SCI), 30 individuals with mild cognitive impairment (MCI), six patients with Alzheimer's dementia (AD), eight patients with frontotemporal dementia (FTD), seven patients with Parkinson's disease (PD), three patients with Lewy body disease (LBD), five patients with vascular MCI (V-MCI), and 13 patients with mixed dementias. Given that ours is, in this embodiment, a study on segmentation performance, applicant can assume that there may be no difference in the T2* appearance of a microbleed related to participants' diagnosis.

Ethical agreements can be obtained for all sites. Participants gave written informed consent before enrollment in the study.

### MRI Acquisition

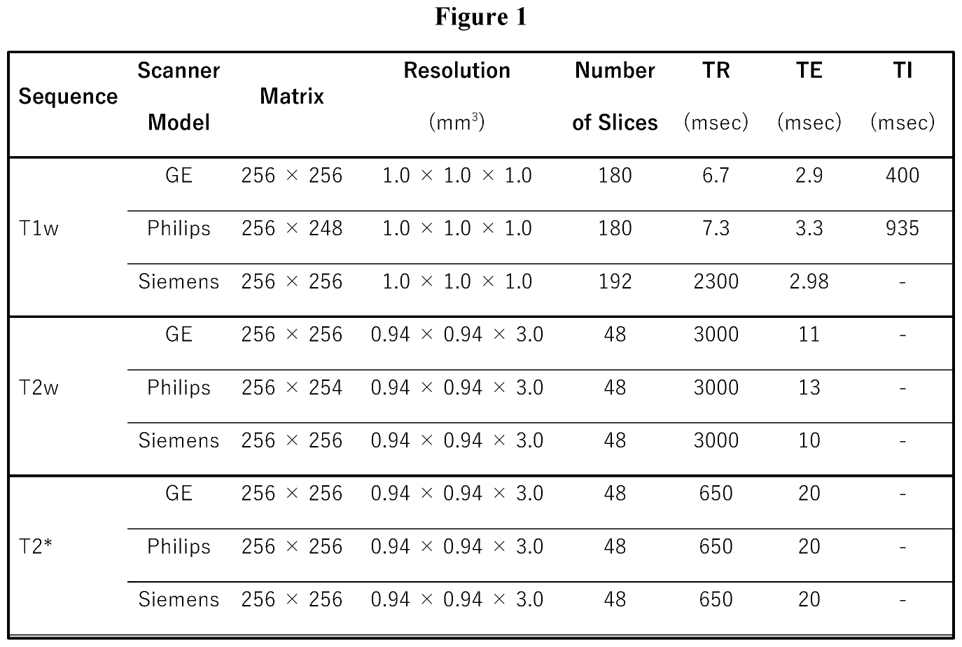

MRI data for all subjects in the CCNA can be acquired with the harmonized Canadian Dementia Imaging Protocol [www.cdip-pcid.ca; (Duchesne et al., 2019)]. FIG. 1 shows a table summarizing the scanner information and acquisition parameters for the subjects included in this exemplary study (acquisition parameters of the harmonized Canadian Dementia Imaging protocol), where TR is the repetition time; TE is the echo time and TI is inversion time.

### MRI Preprocessing

All T1-weighted, T2-weighted and T2* images can be pre-processed as follows: intensity non-uniformity correction (Sled et al., 1998), and linear intensity standardization to a range of [0-100]. Using a 6-parameter rigid registration, the three sequences can be linearly coregistered (Dadar et al., 2018). The T1-weighted images can also be linearly (Dadar et al., 2018) and nonlinearly (Avants et al., 2008) registered to the MNI-ICBM152-2009c average template (Manera et al., 2020). Nonlinear registrations may be performed to generate the necessary inputs for BISON and may not be applied to the data used for training the models. Brain extraction may be performed on the T2* images using BEaST brain segmentation tool (Eskildsen et al., 2012).

### CSF Segmentation

The Brain tISue segmentatiON (BISON) tissue classification tool may be used to segment CSF based on the T1-weighted images (Dadar and Collins, 2020). BISON is an open source pipeline based on a random forests classifier that has been trained using a set of intensity and location features from a multi-center manually labeled dataset of 72 individuals aged from 5-96 years (data unrelated to this study) (Dadar and Collins, 2020). BISON has been validated and used in longitudinal and multi scanner multi-center studies (Dadar et al., 2020; Dadar and Duchesne, 2020; Maranzano et al., 2020).

### Grey and White Matter Segmentation

All T1-weighted images can also be processed using FreeSurfer version 6.0.0 (recon-all-all). FreeSurfer (https://surfer.nmr.mgh.harvard.edu/) is an open source software that provides a full processing stream for structural T1-weighted data (Fischl, 2012). The final segmentation output (aseg.mgz) may then be used to obtain individual masks for cortical GM, deep GM, cerebellar GM, WM, and cerebellar WM based on the FreeSurfer look-up table available at
https://surfer.nmr.mgh.harvard.edu/fswiki/FsTutorial/AnatomicalROI/FreeSurferColorLU T. Since FreeSurfer tends to segment some WMHs as GM (Dadar et al., 2021), Applicant can also segmented the WMHs using a previously validated automated method (Dadar et al., 2017b, 2017a) and used them to correct the tissue masks (i.e. WMH voxels that can be segmented as cortical GM or deep GM by FreeSurfer can be relabeled as WM, and WMH voxels that can be segmented as cerebellar GM by FreeSurfer can be relabeled as cerebellar WM).

### Manual Segmentation

The microbleeds can be segmented by an expert rater (JM>15 years of experience reading research brain MRI) using the interactive software Display package, part of the minc-toolkit (https://github.com/BIC-MNI) developed at the McConnell Brain Imaging Center of the Montreal Neurological Institute. The software can allow visualization of co-registered MRI sequences (T1w, T2w and T2*) in three planes simultaneously, and cycling between sequences, to accurately assess the signal intensity and anatomical location of an area of interest. Identification criteria may be in accordance with Cordonnier et al., 2007, comprised a round area of hypointensity on T2* within the brain tissue and exclusion of colocalization with blood vessels based on T1w and T2w information (Cordonnier et al., 2007). A maximum diameter cut-off point of 10 mm may be use to exclude large hemorrhages (Cordonnier et al., 2007). In this embodiment, no minimum microbleed size cut-off may be used. Eight cases with varying number of microbleeds can be segmented a second time by the same rater to assess intra-rater variability.

### Quality Control

The quality of the preprocessed images may be visually assessed, as well as the BISON and FreeSurfer automated segmentations.

### Generating Training Data

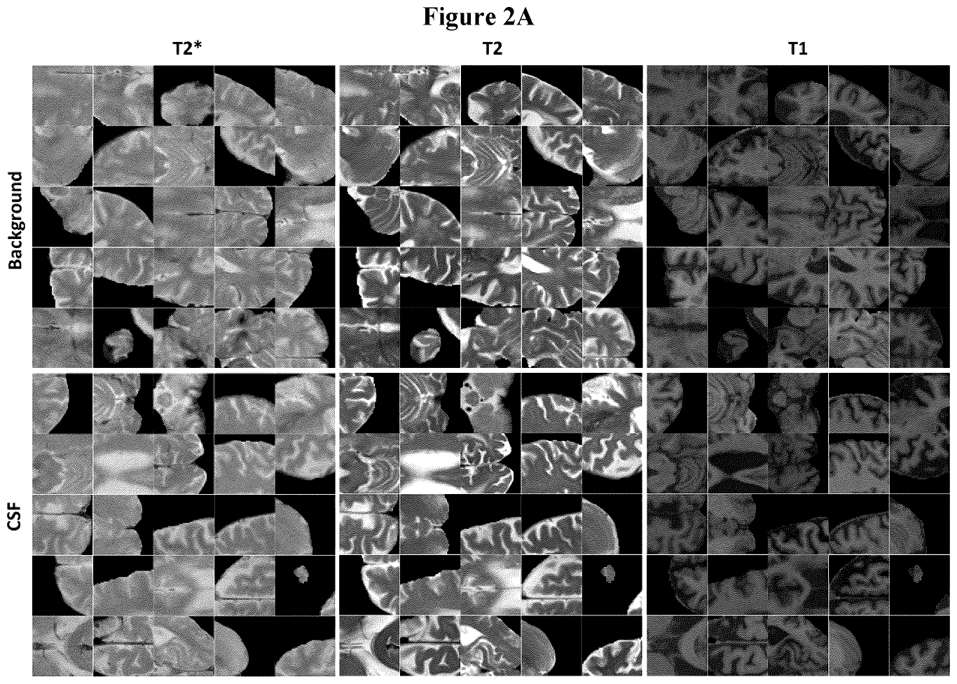

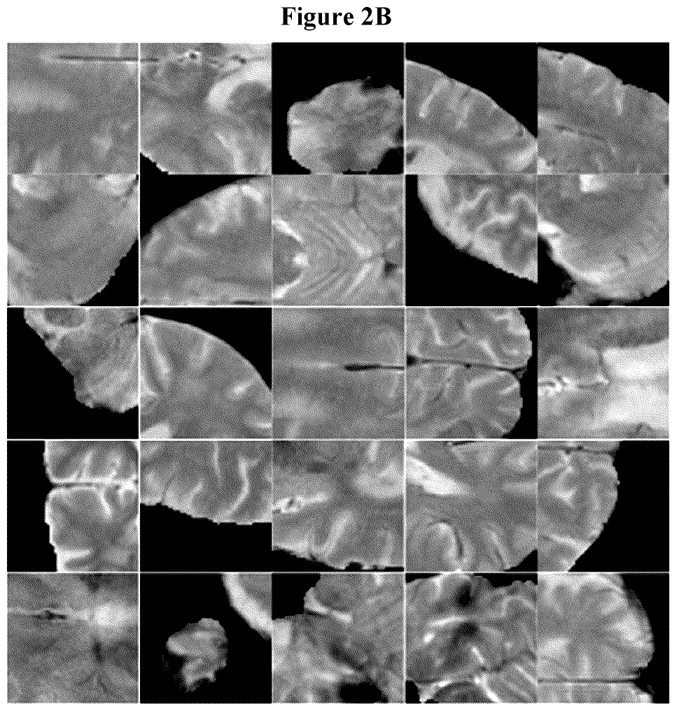

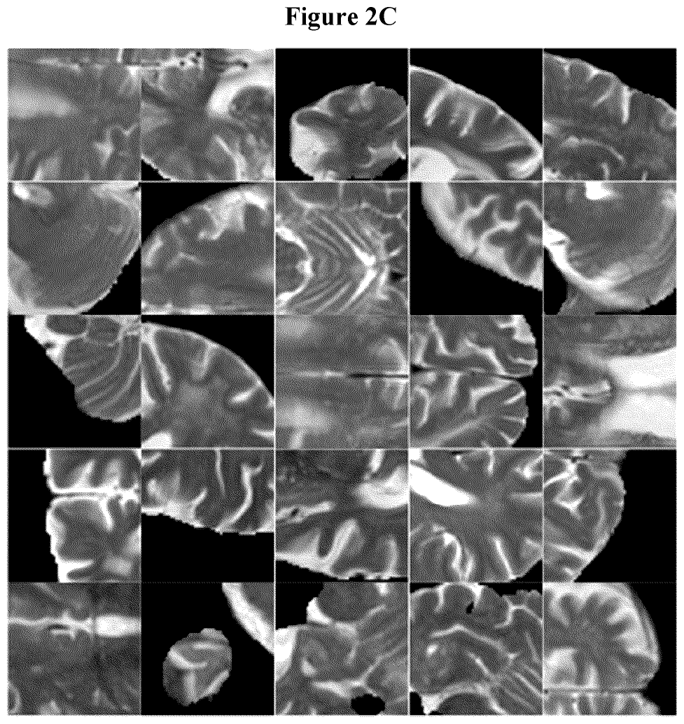

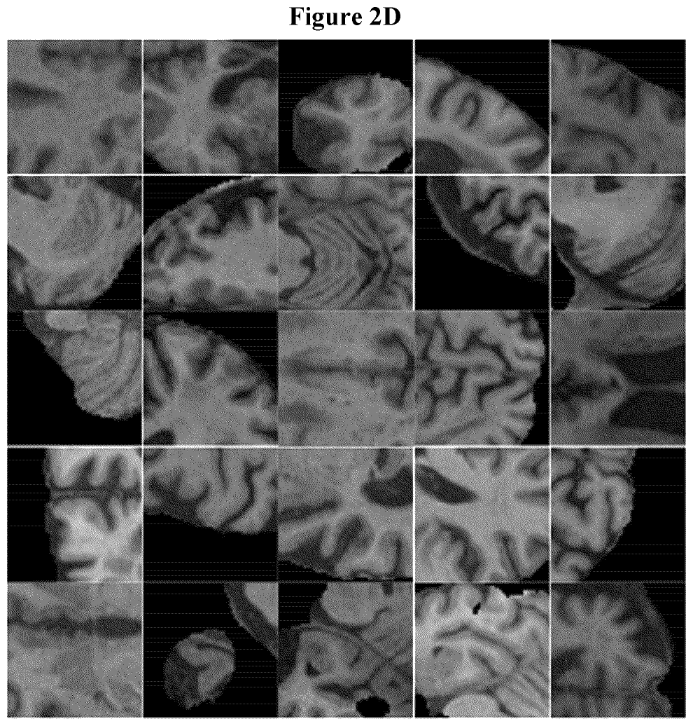

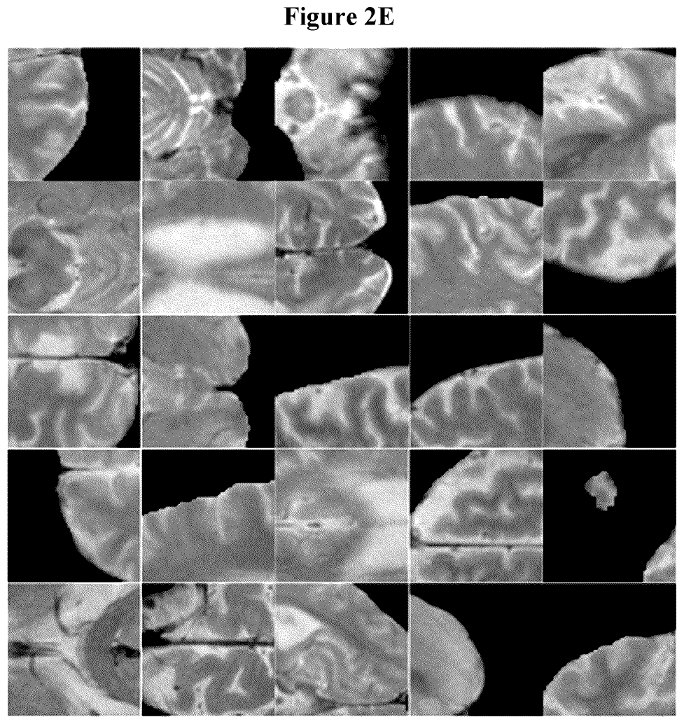

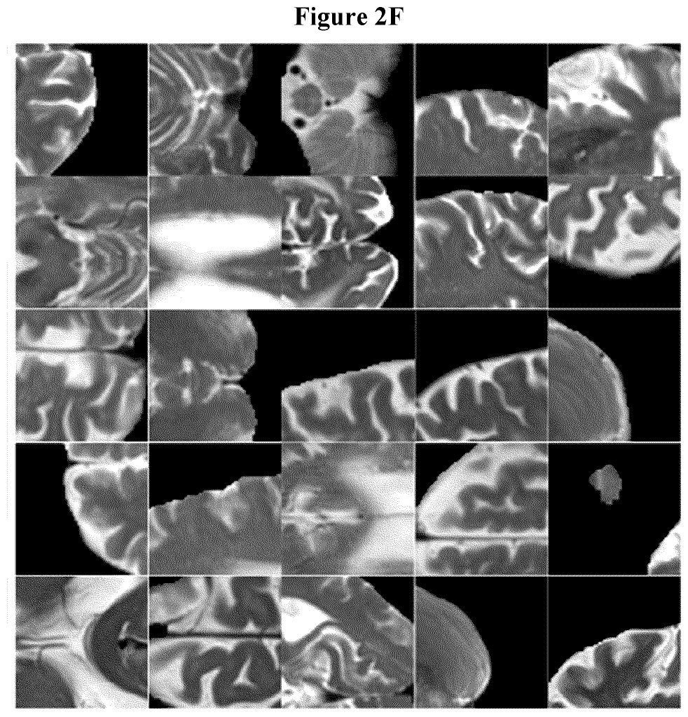

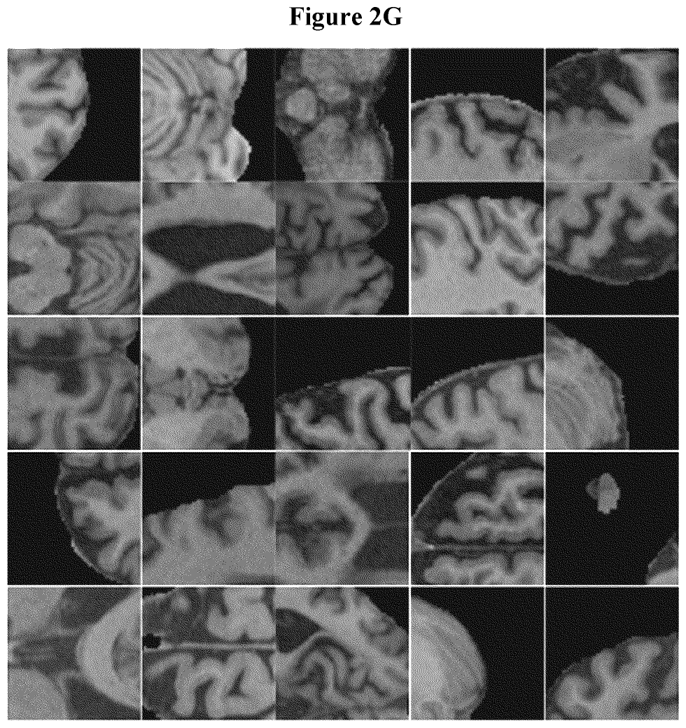

CSF Segmentation Task: 400,000 randomly sampled two-dimensional (2D) image patches can be generated from the in-plane (axial plane, i.e. the plane with the greatest resolution) preprocessed and co-registered T2*, T2-weighted, and T1-weighted image slices. Half of the generated patches contained a voxel segmented as CSF by BISON in the center voxel of the patch, and the other half contained either GM or WM in the center of the patch. The patches can be randomly assigned to training, validation and test sets (50%, 25%, and 25% respectively). To avoid leakage, patches that can be generated from one participant that is only included in the same set; i.e. the random split may be performed at participant level (Mateos-Pérez et al., 2018). FIG. 2A shows examples of the generated CSF and background patches and FIGS. 2B to 2G are enlarged images of the subsets of FIG. 2A (in order from left to right and top to bottom).

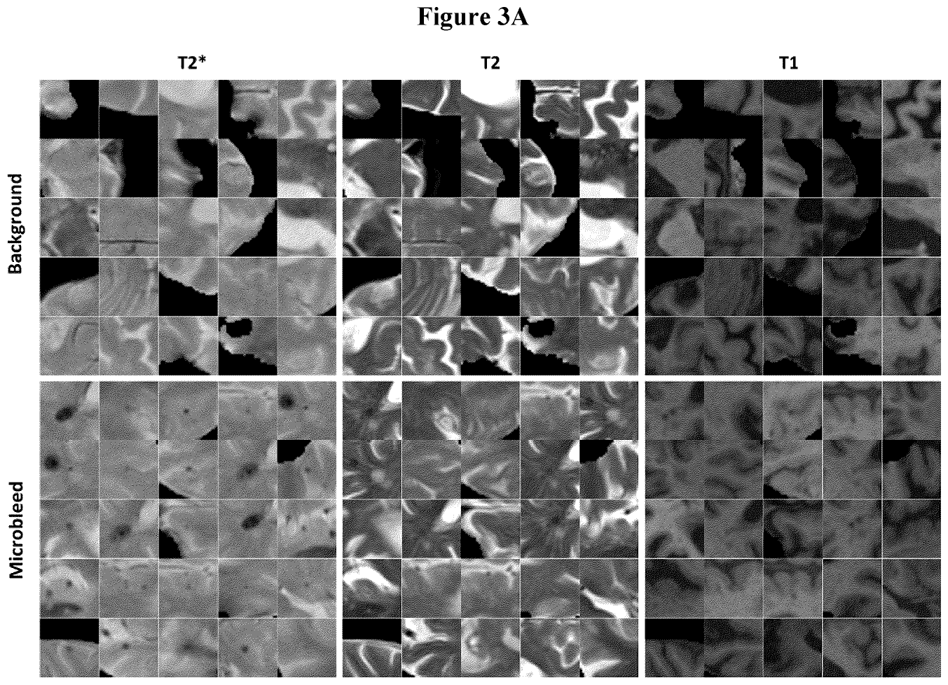

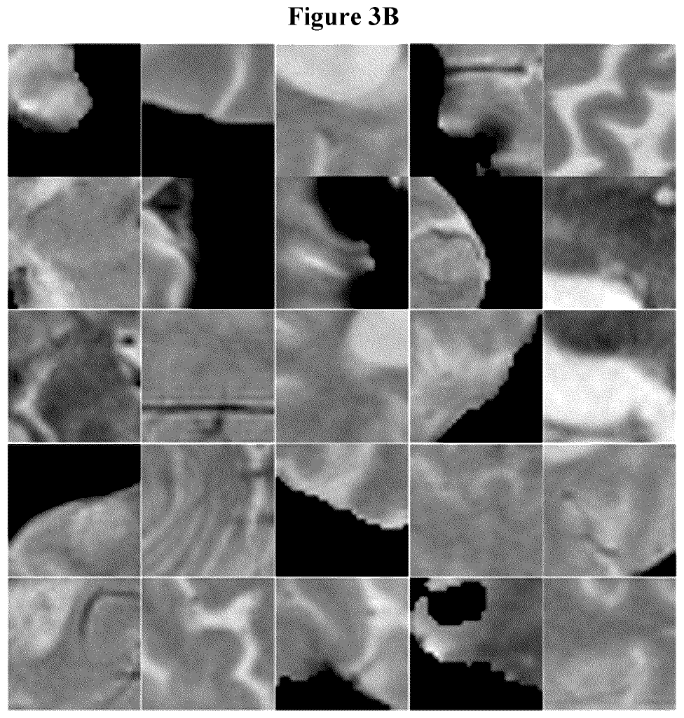

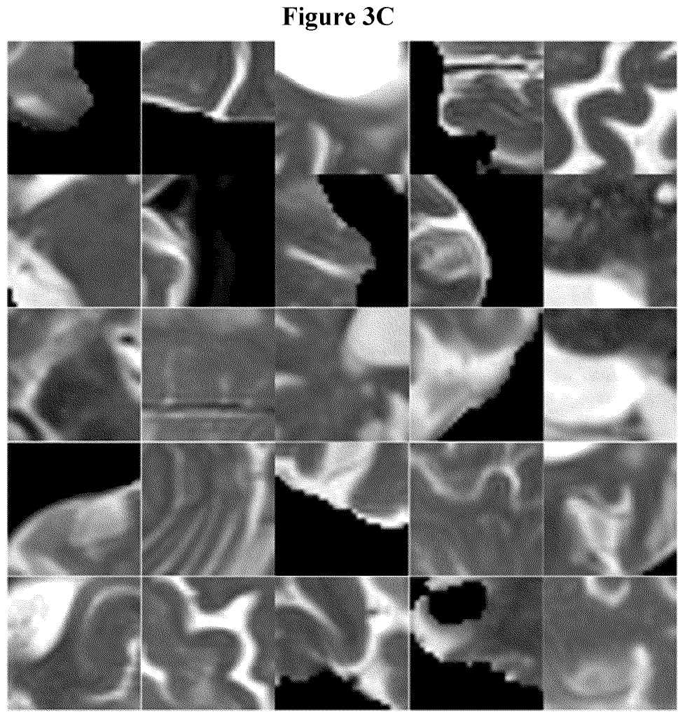

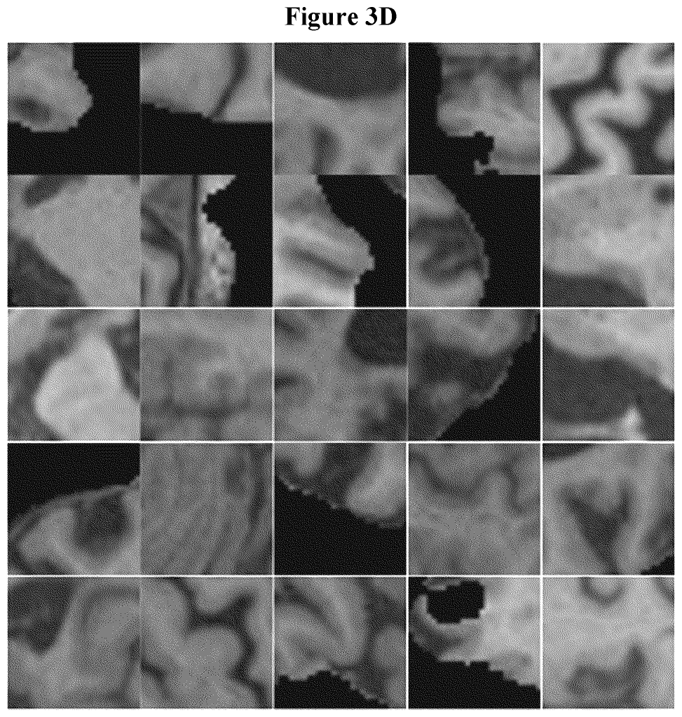

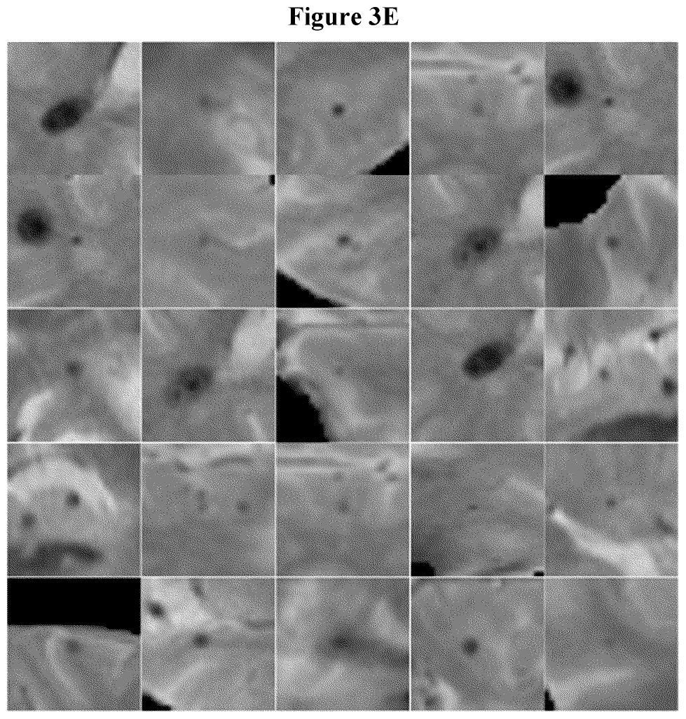

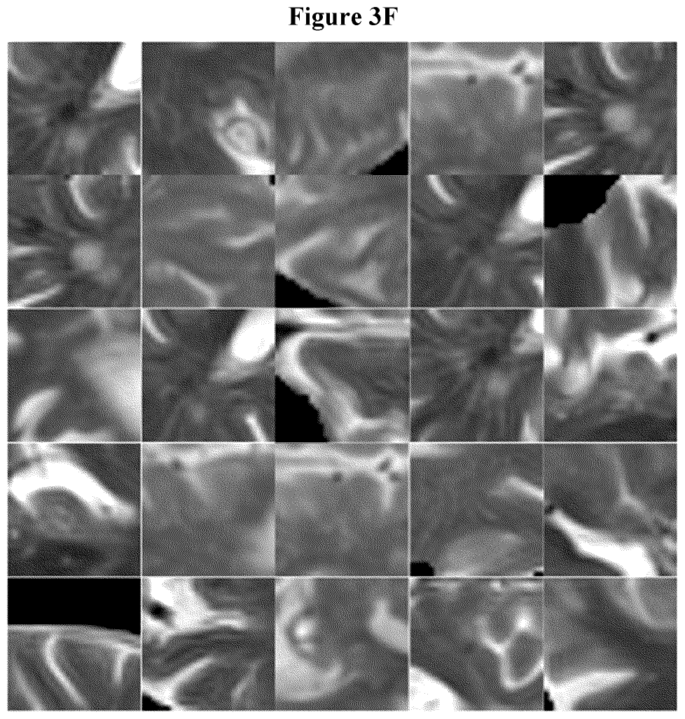

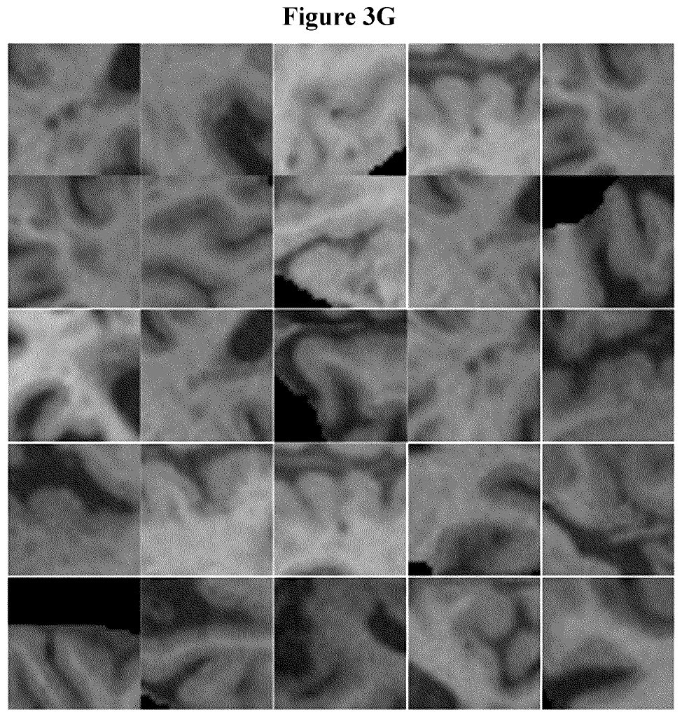

Microbleed Segmentation Task: Similarly, 11,570 2D patches can be generated from the preprocessed and co-registered T2*, T2-weighted, and T1-weighted in-plane image slices for microbleed segmentation. Half of the generated patches contained a voxel segmented as microbleed by the expert rater in the center voxel of the patch, and the other half may be randomly sampled to contain a non-microbleed voxel in the center. The patches may be randomly assigned to training, validation and test sets (60%, 20%, and 20% respectively) also at the participant level. Applicant can further ensured to include similar proportions of participants with small (1-4 voxels), medium (5-15 voxels), and large (more than 15 voxels) microbleeds in the three sets. FIG. 3A shows examples of the generated microbleed and background patches and FIGS. 3B to 3G are enlarged images of the subsets of FIG. 3A (in order from left to right and top to bottom).

Applicant further augmented the microbleed patch dataset by randomly rotating the patches to generate additional training data. The random rotations may be performed on the full slice (not the patches) centering around the microbleed voxel; therefore, the corner voxels in the patches include information from different areas not present in other patches. Matching numbers of novel background patches may also be added to balance the training dataset. The performance of the model may be assessed using the training dataset with no augmentation, and with adding 4, 9, 14, 19, 24, and 29 random rotations to the training set, respectively.

Note that the sameMRI dataset may be used to generate patches for both CSF segmentation and microbleed segmentation tasks. However, the individual patches may not be the same, since the microbleed and CSF patches needed to have (respectively) either a microbleed or CSF voxel at their center, while the background patches may be randomly sampled from the rest of the dataset.

Training the ResNet50 Network using Transfer Learning

Applicant can use the ResNet50 network (He et al., 2016), a CNN pre-trained on over 1 million images from the ImageNet dataset (Challenge, 2012) to classify images into 1000 object categories. This pre-training has allowed the network to learn rich feature representations for a wide range of images, which can also be useful in our task of interest. Our approach may be to further train ResNet50 first on a task similar to microbleed segmentation (i.e. CSF vs. tissue) then on microbleeds identification itself.

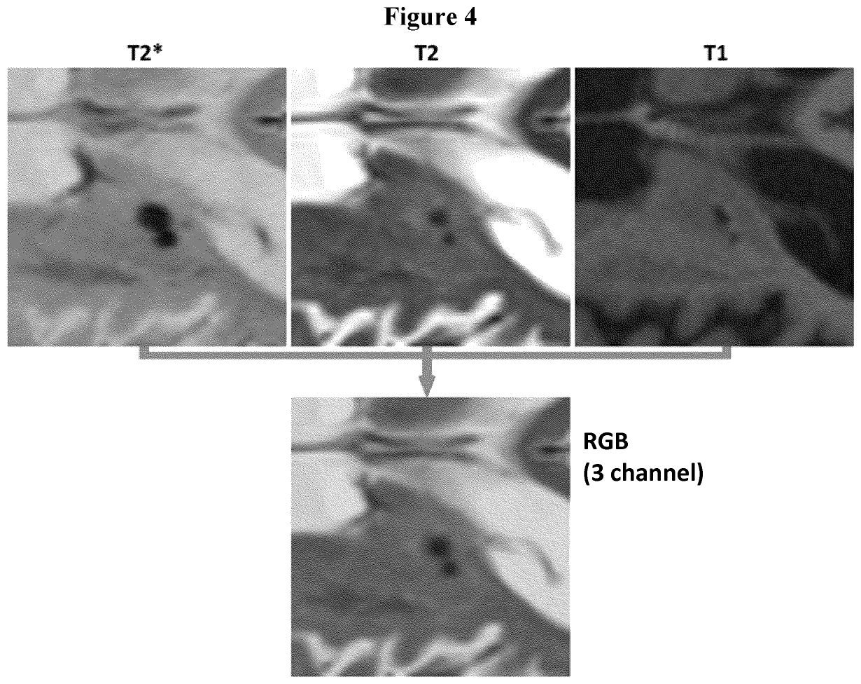

To satisfy the input size requirements of ResNet50 network, all patches may be resized to 224×224 pixels, and the T2*, T2-weighted, and T1-weighted patches may be copied in to three channels to generate an RGB image (FIG. 4). The last fully connected layer of ResNet50 which contained 1000 neurons (to perform the classification task for 1000 object categories) may be replaced with two neurons to adapt the network for performing a binary classification task (i.e. object versus background). The weights of this last fully connected layer may be initialized randomly. The network can first be trained (all layers, no weight freezing) to perform the CSF versus tissue segmentation task. Applicant then retrained this network to perform microbleed versus tissue segmentation. Training can be performed on a single NVIDIA TITAN X with 12 GB GPU memory.

### Parameter Optimization

Applicant assessed the performance of the network with five different patch sizes (14, 28, 52, 56, and 70 mm²), with and without freezing the weights of the initial layers (no freezing as well as freezing the first 5, 10, 15, and 20 layers), different mini-batch sizes (20, 40, 60, 80, and 100), and different learning rates (0.002, 0.004, 0.006, 0.008, and 0.010). Each experiment may be repeated five times by changing one hyper-parameter at a time, and the results may be averaged to ensure their robustness. Epoch parameter may be set to 10. Stochastic gradient descent with momentum (SGDM) of 0.9 may be used to optimize the cross-entropy loss.

### Post-Processing

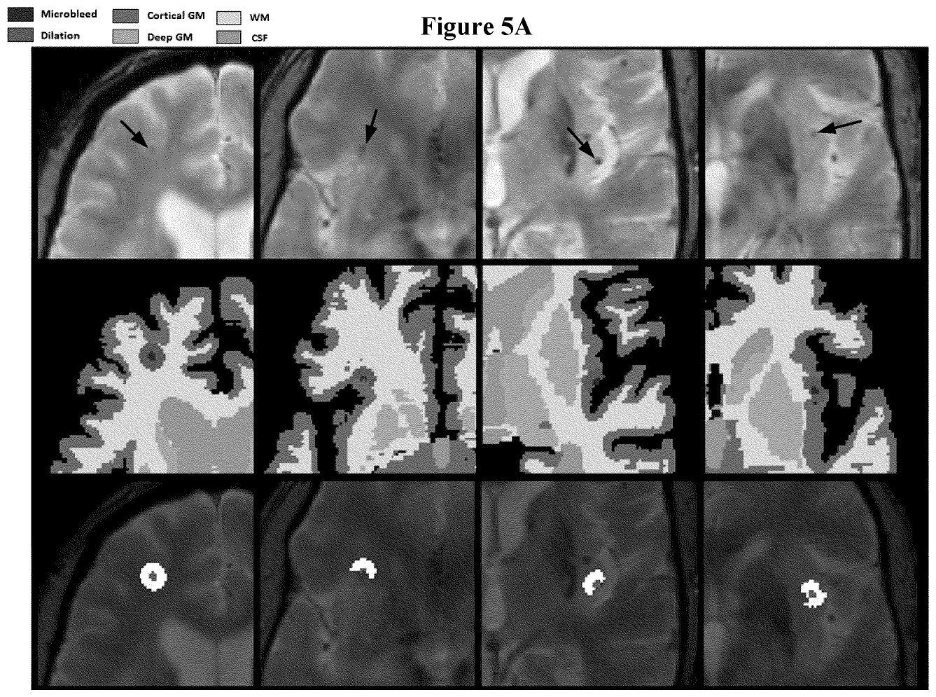

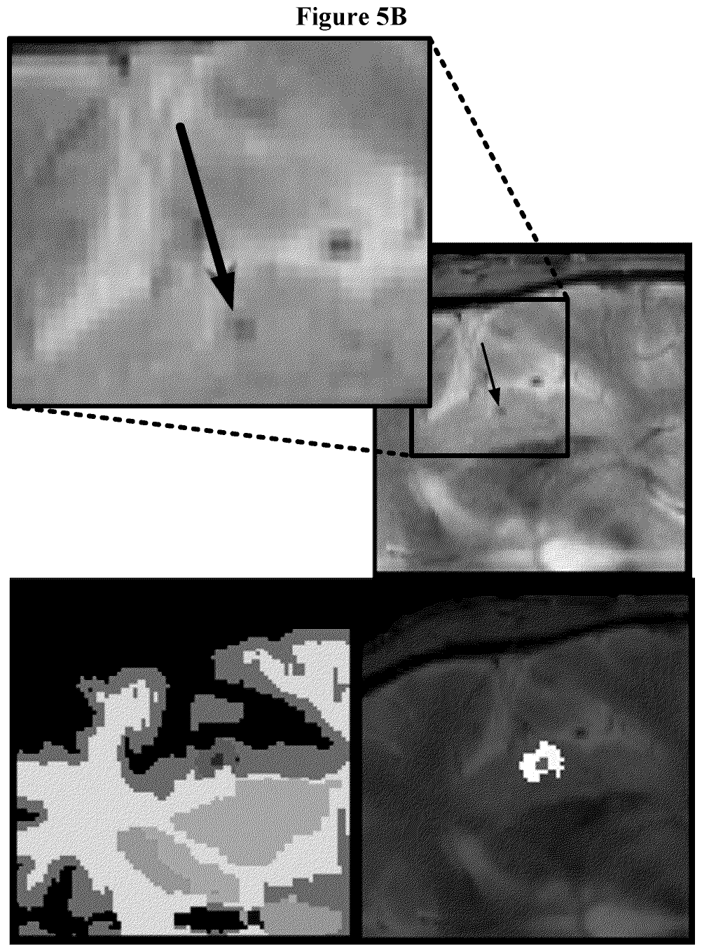

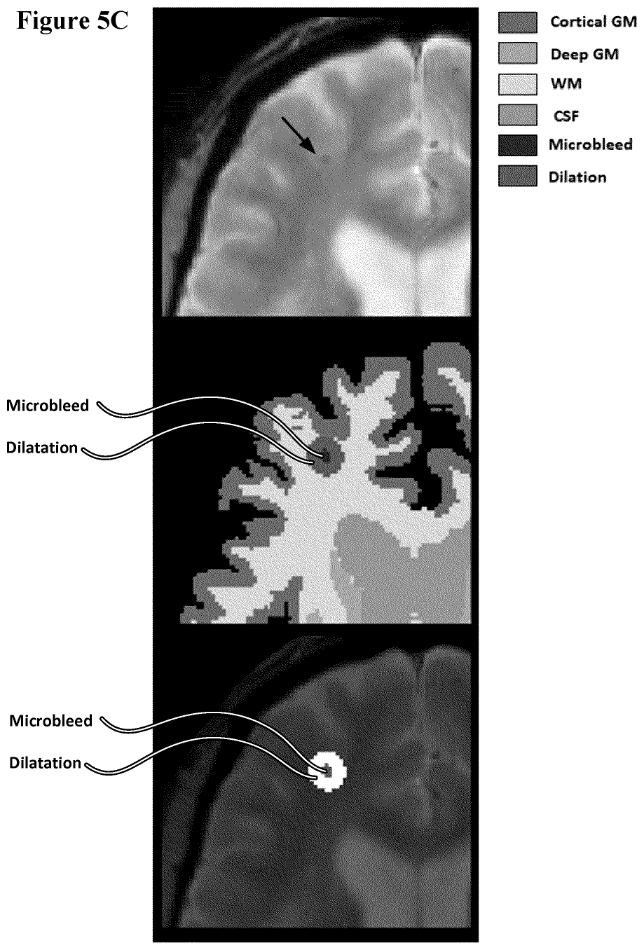

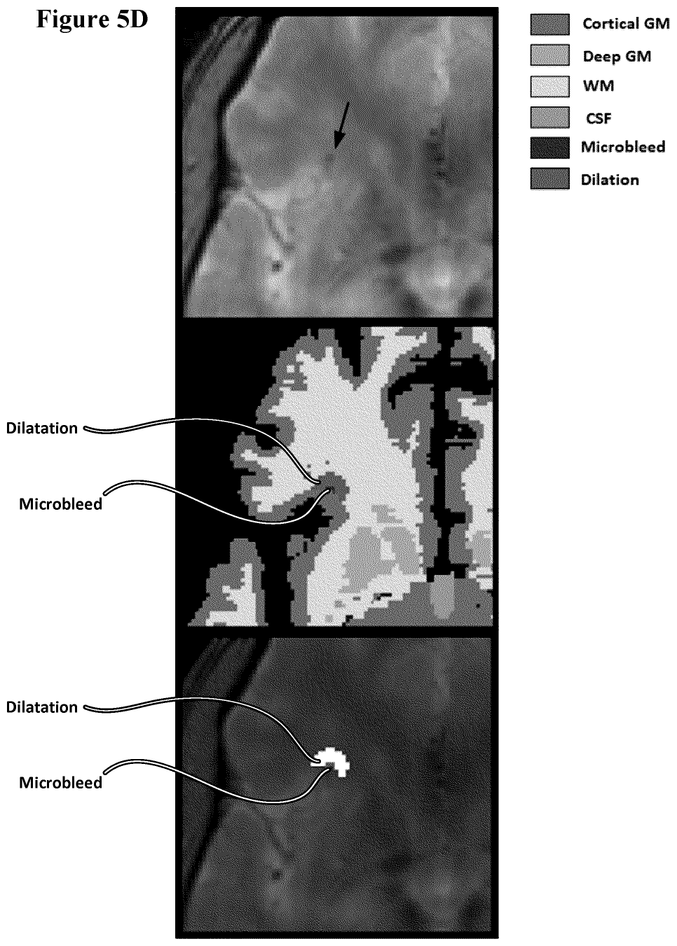

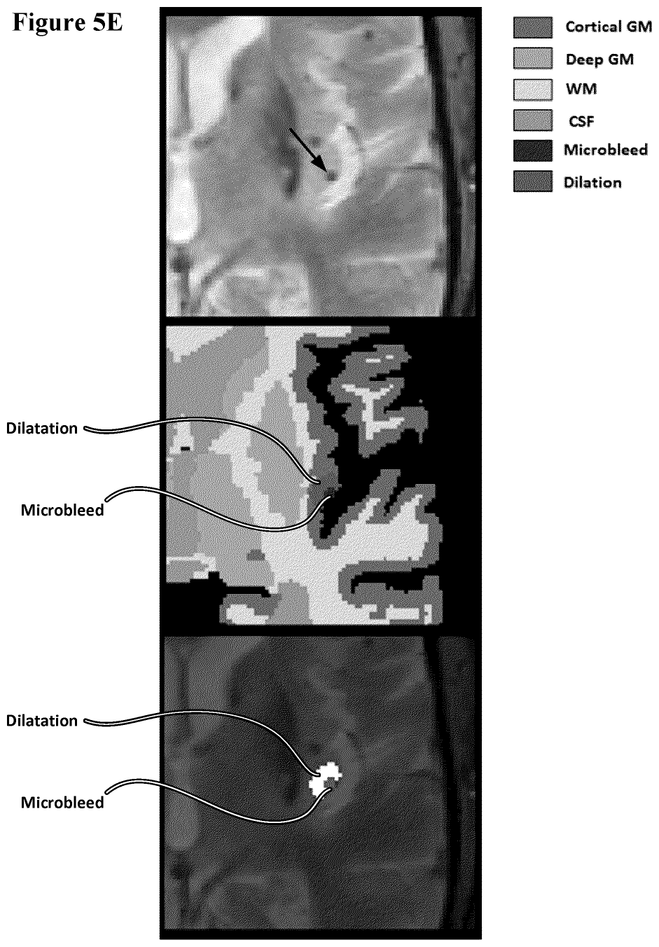

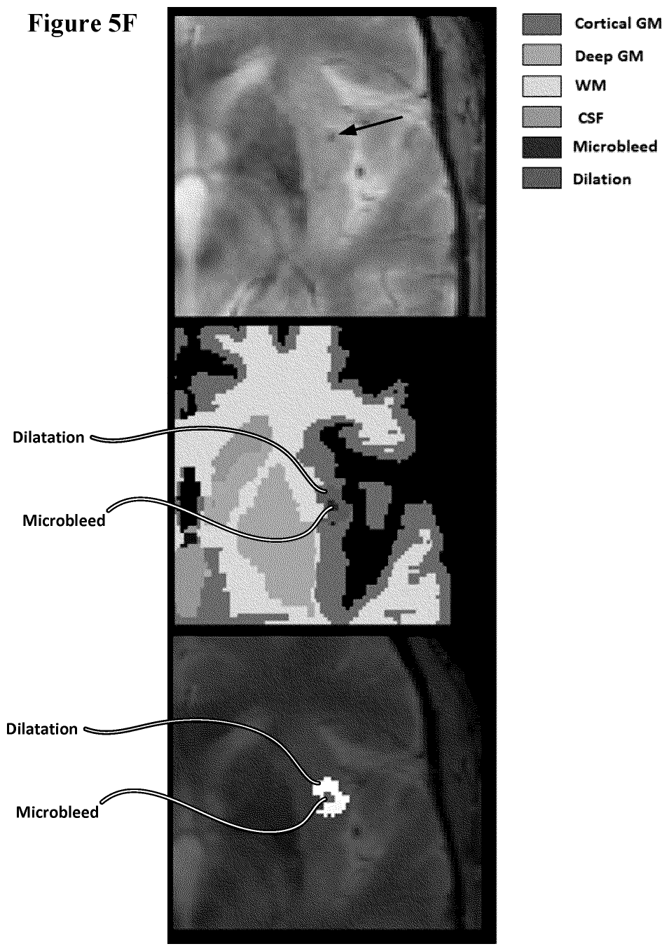

After applying ResNet50 to segment all microbleed candidate voxels in the brain mask and reconstructing the final map into a 3D segmentation map, applicant performed a post-processing step to reduce the number of false positives as well as to categorize the microbleeds into five classes depending on location. In this post-processing step, the microbleeds may first be dilated by two voxels. Then, for each voxel at the border of the microbleed, if the ratio between the T2* intensity of the microbleed voxel and the surrounding dilated area (Microbleed Intensity/Dilated Mask Intensity) may be lower than a specific threshold (to be specified by the user based on sensitivity/specificity preferences), the voxel may be removed as a false positive. For each microbleed, the process may be repeated iteratively until no voxel can no longer be removed as false positive. The final remaining microbleeds can then be categorized into regions (cortical GM, deep GM, cerebellum WM, and cerebellum GM and WM based on their overlap with FreeSurfer segmentations). FIG. 5A shows examples of FreeSurfer based tissue categories, some segmented microbleeds and dilated surrounding areas. FIG. 5C shows axial slices showing FreeSurfer based tissue categories, segmented microbleeds and their dilated surrounding areas (zoomed-in from FIG. 5A). FIG. 5D shows axial slices showing FreeSurfer based tissue categories, segmented microbleeds and their dilated surrounding areas (zoomed-in from FIG. 5A). FIG. 5E shows axial slices showing FreeSurfer based tissue categories, segmented microbleeds and their dilated surrounding areas (zoomed-in from FIG. 5A). FIG. 5F shows axial slices showing FreeSurfer based tissue categories, segmented microbleeds and their dilated surrounding areas (zoomed-in from FIG. 5A).

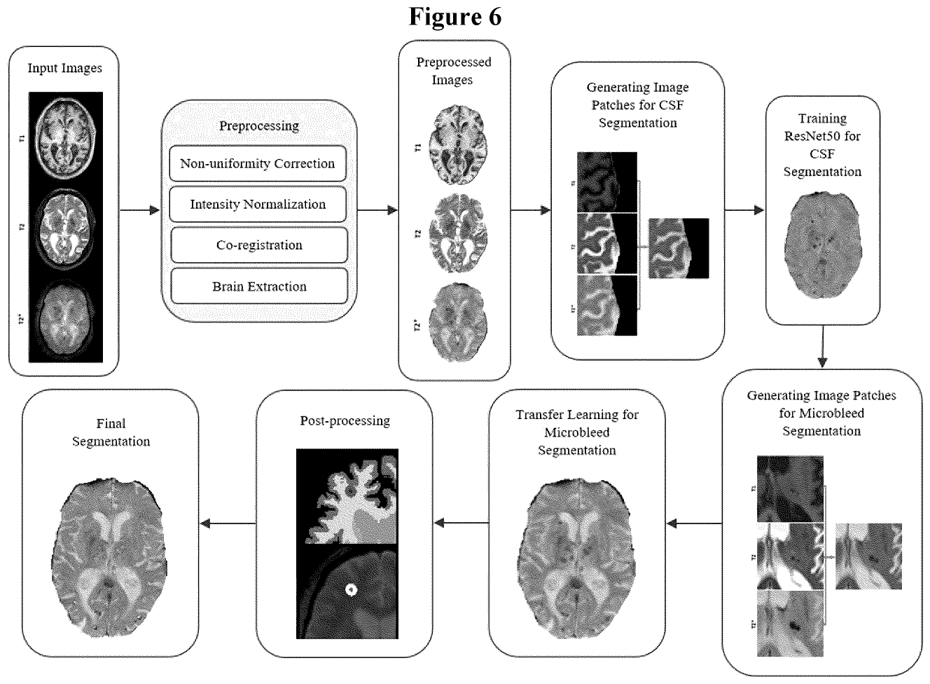

FIG. 6 shows a flow diagram of all the different steps performed in the microbleed segmentation pipeline. All implementations (i.e. generating training patches, training and validation of the model, and postprocessing) may be performed using MATLAB version 2020b.

### Performance Validation

At patch level, to enable comparisons against results of other studies, applicant measured accuracy, sensitivity, and specificity to assess the performance of the proposed method.

$$Accuracy = \frac{TP + TN}{TP + TN + FP + FN} ; Sensitivity = \frac{TP}{TP + FN} ; Specificity = \frac{TN}{TN + FP}$$

Where TP, TN, FP, and FN denote the number of true positive, true negative, false positive, and false negative patch-level classifications, respectively. While high accuracy at patch-level can be desirable, it may not necessarily ensure accurate segmentations at a voxel level. Applied across the entire brain (i.e. 100,000s of pixels), a patch-level specificity of 0.96-0.99% (e.g. as reported by Hong et al. (Hong et al., 2020, 2019)) might translate into thousands of false positive voxels. To assess the performance at a voxel-wise level, applicant applied the network to all patches in the brain mask, reconstructed the lesions in 3D across patches, and then measured per lesion sensitivity, precision, and Dice similarity index (Dice, 1945) values between manual (considered as the standard of reference) and automated segmentations.

$$Sensitivity = \frac{TP}{TP + FN} ; Precision = \frac{TP}{TP + FP} ; Dice Similarity Index = 2 \frac{MB 1 ⋂ MB 2}{MB 1 + MB 2}$$

Here, TP (true positive) denotes the number of microbleeds (i.e., the number of distinct microbleeds segmented, regardless of their size) detected by both methods. FN (false negative) denotes the number of microbleeds detected by the manual expert, but not the automated method. FP (false positive) denotes the number of microbleeds detected by the automated method, and not the manual rater. Dice similarity index shows the proportion of microbleeds detection by both methods, over the number of microbleeds detected by each method. A Dice Similarity index of 1 shows perfect agreement between the two methods. A microbleed can be considered to be detected by both methods if there are any overlapping microbleed voxels between the two segmentations. Note that in accordance with previous studies, specificity may be used to reflect the proportion of true negative versus all negative classifications for patch-level results. However, since specificity cannot be defined at per-lesion level, applicant assessed precision instead of specificity for per-lesion results.

### U-Net Segmentations

In accordance with an embodiment, a U-Net model was also trained on complete axial slices (256 mm2) as well as smaller patches (patch sizes of 32, 64, 128 mm2) from the same dataset for comparison (SGDM, Epoch=10, initial learning rate=0.001, mini-batch size=20). The input images may be similar to those used for the ResNet50 model (i.e., T2*, T1-weighted, and T2-weighted axial slices), and the same number of training samples as those used to train the best ResNet50 models (i.e., with data augmentation) may be generated for the segmentation tasks. The performance of the model in detecting the microbleeds (i.e., per lesion sensitivity) may be then assessed for different patch sizes at the patch level. For the optimal patch size, the voxel level performance vs. the testing time can also be assessed for overlapping patches (using the labels from the centering voxels of each patch) with different stride values from 1, ½, ¼, ⅛, and 1/16 of the patch sizes to investigate whether performance improves when using overlapping patches at the expense of longer processing time.

### Data and Code Availability Statement

All image processing steps can be performed using minc tools, publicly available at: https://github.com/vfonov/nist_mni_pipelines. BISON (used for tissue classification) is also publicly available at http://nist.mni.mcgill.ca/?p=2148. For more information on CCNA dataset, please visit https://ccna-ccnv.ca/.

### BISON

BISON is a tissue classification tool publicly available at: http://nist.mni.mcgill.ca/tissue-classification/Citation of Dadar, Mahsa, and D. Louis Collins: “BISON: Brain tissue segmentation pipeline using T1-weighted magnetic resonance images and a random forest classifier.” Magnetic Resonance in Medicine 85.4 (2021): 1881-1894.

To perform tissue segmentation using BISON, the user needs to install minc tools (available at http://bic-mni.github.io/), as well as Python 2.7.13: : Anaconda 2.5.0 (64-bit). The pipeline requires a list providing the path to the T1w MR images to be segmented as input. The user can run BISON using the following command:
python BISON.py (-c RF-m Trained_Classifiers/-o Outputs/-t Temp_Files/-e PT-n List.csv-p Trained_Classifiers/-1 3-c RF).
Determines that a random forest classifier will be used for segmentation-m Trained_Classifiers/
Determines the path to the prior models-o Outputs/
Determines the path to the outputs that will be generated by BISON-t Temp_Files/
Determines the path to a folder that will be used to save temporary files-e PT
Determines the use of pre-trained classifiers-p Trained_Classifiers/
Determines the path to the pre-trained classifiers-1 3
Determines the number of classes to be segmented-n List.csv
Determines the path to a list of the input files to be segmented

### SegMB

SegMB is the developed microbleed segmentation tool. To perform microbleed segmentation using SegMB, the user requires minc tools (available at http://bic-mni.github.io/), as well as MATLAB. The pipeline requires a list providing the path to the T1w, T2w, and T2* MR images to be segmented as input. The user can run SegMB using the following command:
SegMB (path_trained_net, path_input_csv, path_output) path_trained_net
Determines the path to the pre-trained deep learning network path_input_csv
Determines the path to a list of the input files to be segmented path_output
Determines the path to the outputs that will be generated by SegMB

### Post-Processing

After applying ResNet50 to segment all microbleed candidate voxels, applicant performed a post-processing step to reduce the number of false positives as well as to categorize the microbleeds into five classes depending on location. In this post-processing step, the microbleeds can first be dilated by two voxels. Then, for each voxel at the border of the microbleed, if the ratio between the T2* intensity of the microbleed voxel and the surrounding dilated area [Microbleed Intensity/(Dilated Mask Intensity)] may be lower than a specific threshold (to be specified by the user based on sensitivity/specificity preferences), the voxel may be removed as a false positive. For each microbleed, the process may be repeated iteratively until no voxel may be removed as false positive. The final remaining microbleeds can then be categorized into regions (cortical GM, deep GM, cerebellum WM, and cerebellum GM and WM based on their overlap with FreeSurfer segmentations). FIG. 4 shows examples of FreeSurfer based tissue categories, some segmented microbleeds and dilated surrounding areas.

It will be appreciated that the comparison made by calculating the ratio can alternatively be performed using statistical or machine learning approaches (and for greater clarity, this includes deep learning).

The following is an example of computer program code able to perform the post-processing (while permission can be granted to reproduce the listing as part of this patent specification, Applicant does not waive any other copyright to this listing):

```
function post_processing(path_output,Info,postp_flag,ID,img,mb,brain)
addpath(genpath(‘/data/ipl/proj13/WMH_Data/niak’)); % Used to read minc files
if postp_flag == 1
  %% Minimum and maxmimum acceptable microbleed size, for post processing
  min_microbleed_size=1;
  max_microbleed_size=1000;
  [~,S]=niak_read_minc(cell2mat(strcat(path_output,‘/’,Info.IDs(ID),{‘_FS_cr_t2star.mnc’})));
  %%
  ind_gm_cortical=[3;17;18;19;28;42;53;54;60;80];
  ind_gm_basal_ganglia=[9;10;11;12;13;26;48;49;50;51;52;58];
  ind_gm_cerebellum=[6;8;47;45];
  ind_wm=[2;41;77;251;252;253;254;255];
  ind_wm_cerebellum=[7;16;46;85];
  ind_csf=[4;5;14;15;24;30;31;43;44;62;63;72];
  M=S*0;
  for j=1:size(ind_gm_cortical);M(S==ind_gm_cortical(j))=1;end
  for j=1:size(ind_gm_basal_ganglia);M(S==ind_gm_basal_ganglia(j))=2;end
  for j=1:size(ind_gm_cerebellum);M(S==ind_gm_cerebellum(j))=3;end
  for j=1:size(ind_wm);M(S==ind_wm(j))=4;end
  for j=1:size(ind_wm_cerebellum);M(S==ind_wm_cerebellum(j))=5;end
  for j=1:size(ind_csf);M(S==ind_csf(j))=6;end
  brain2=M>0;
  %%
  fs=M;
  mb(img>median(img(fs==1)))=0;
  mbtt=0*mb;
  for j=1:size(img,3)
    for tt=1:6 % tt: tissue type index
      %% Thresholds
      if tt==2
        th=10;% Postprocessing threshold for deep GM, can be adjusted depending on desired
sensitivity level
      else
        th=3;% Postprocessing threshold for other regions, can be adjusted depending on desired
sensitivity level
      end
      tmpImg=zeros(size(img,1),size(img,2));
      tmpMB=zeros(size(img,1),size(img,2));
      tmpBrain=zeros(size(img,1),size(img,2));
      tmpFS=zeros(size(img,1),size(img,2));
      tmpImg(:,:)=img(:,:,j);tmpFS(:,:)=fs(:,:,j);
      tmpMB(:,:)=mb(:,:,j).*(tmpFS==tt);
      tmpBrain(:,:)=brain(:,:,j).*(tmpFS==tt);
      [labeledImageA, numberOfObjectA] = bwlabeln(tmpMB); % Identifies the unique
components (microbleeds) in the segmentation
      for m=1:numberOfObjectA
        if min(tmpImg(labeledImageA==m))>median(img(fs>0 & fs<6))
          tmpMB(labeledImageA==m)=0;crMB=tmpMB*0;
        else
          crMB=labeledImageA==m;nv_mb=sum(crMB(:));nv_mbnew=0;
          while nv_mb>nv_mbnew % iterative removal of false positives
            nv_mb=sum(crMB(:));nv_mbnew=0;
            se=strel(‘sphere’,1);crMBd=crMB−
imerode(crMB,strel(‘sphere’,1));ind_crMBd=find(crMBd);
            se1=strel(‘sphere’,4);se2=strel(‘sphere’,2);
            dl=(imdilate(crMB,se2)−crMB).*tmpBrain;
            dl2=(imdilate(crMB,se2)−crMB);
            for n=1:size(ind_crMBd,1)
              if
(min(median(tmpImg(dl>0)),median(tmpImg(dl2>0)))/tmpImg(ind_crMBd(n)))<(0.95+th/20)
                crMB(ind_crMBd(n))=0;dl=(imdilate(crMB,se2)−
crMB).*tmpBrain;dl2=(imdilate(crMB,se2)−crMB);
              end
            end
            nv_mbnew=sum(crMB(:));
          end
        end
        tmpMB(labeledImageA==m)=crMB(labeledImageA==m);
      end
      mbtt(:,:,j)=mbtt(:,:,j)+tmpMB*tt; % assigns a tissue-specific label to the post-processed
microbleed
    end
  end
  [labeledSeg, numberOfMBs] = bwlabeln(mbtt>0);
  for i = 1 : numberOfMBs % removing microbleeds that are larger or smaller than the predefined
acceptable size
    if sum(labeledSeg(:)==i)<min_microbleed_size
      mbtt(labeledSeg==i)=0;
    end
    if sum(labeledSeg(:)==i)>max_microbleed_size
      mbtt(labeledSeg==i)=0;
    end
  end
  % Generating a summary table for the microbleeds
  Summary=table;
  [labeledSeg, Summary.N_gm_cortical] = bwlabeln(mbtt==1);
  [labeledSeg, Summary.N_gm_basal_ganglia] = bwlabeln(mbtt==2);
  [labeledSeg, Summary.N_gm_cerebellum] = bwlabeln(mbtt==3);
  [labeledSeg, Summary.N_wm] = bwlabeln(mbtt==4);
  [labeledSeg, Summary.N_wm_cerebellum] = bwlabeln(mbtt==5);
  [labeledSeg, Summary.N_csf] = bwlabeln(mbtt==6);
  [labeledSeg, Summary.N_total] = bwlabeln(mbtt>0);
  writetable(Summary,cell2mat(strcat(path_output,‘/’,Info.IDs(ID),‘_MicroBleed_Summary.csv’)))
  % Saving the post-processed segmentation mask
  hdr.file_name=cell2mat(strcat(path_output,‘/’,Info.IDs(ID),‘_MicroBleed_PP.mnc’));
  niak_write_minc(hdr,mbtt);
end
```

### RESULTS

### Manual Segmentation and Quality Control

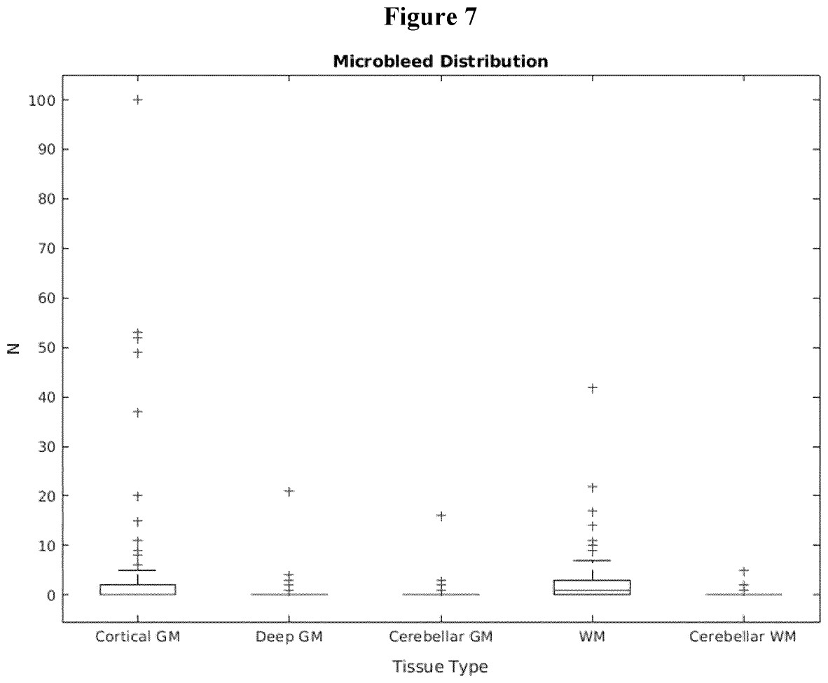

The distribution of manually segmentated lesions for all 78 participants can be shown in FIG. 7. Based on the manual segmentations, 46.1%, 14.10%, and 58.9% of the cases had at least one microbleed in the cortical GM, deep GM, and WM regions, respectively. Only five cases (6.4%) had microbleeds in the cerebellum (in either GM and WM). In this embodiment, the overall intra-rater reliability (per lesion similarity index) for manual segmentation was 0.82±0.14 (κ_{Cortical GM}=0.78±0.22, κ_{Deep GM}=1.0±0.0, κ_{WM}=0.81±0.15).

All MRIs passed the visual quality control step for pre-processing and BISON/FreeSurfer segmentation.

### CSF Segmentation

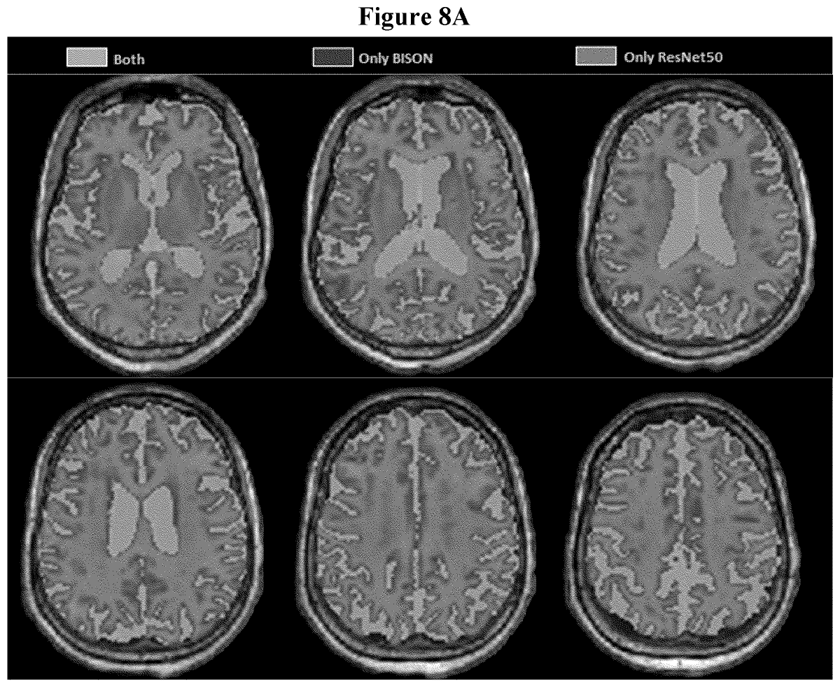

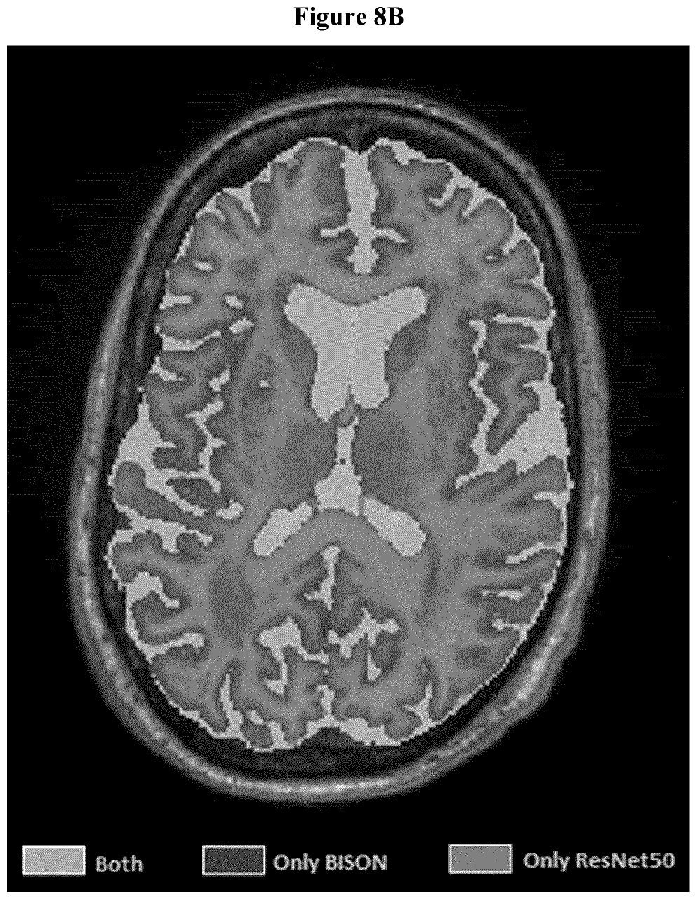

At patch level, ResNet50 segmentations had accuracies of 0.946 (sensitivity=0.955, specificity=0.936) and 0.933 (sensitivity=0.938, specificity=0.928) for validation and test sets, respectively. At a whole brain voxel level, the segmentations had a Dice similarity index of 0.913±0.015 with BISON segmentations. Overall, ResNet50 CSF segmentations (mean volume=129.06±31.43 cm³) were more generous in comparison with BISON (mean volume=117.78±29.51 cm³). FIG. 8A compares the two segmentations, with color yellow indicating voxels that were segmented as CSF with both methods, and colors purple and green indicating voxels that were only segmented as CSF by BISON or ResNet50, respectively. The majority of the disagreements are in the borders of CSF and brain tissue, where ResNet50 segments CSF slightly more generously than BISON.

### Microbleed Segmentation

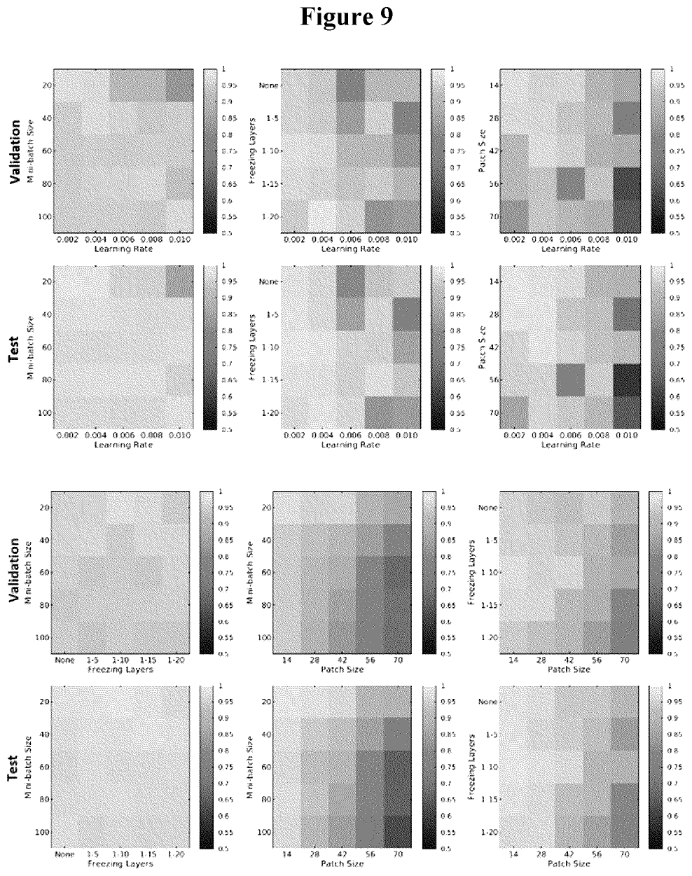

FIG. 9 shows the patch-level performance results averaged over five repetitions for different patch sizes, freezing of initial layers, mini-batch sizes, and learning rates. Increasing patch size to more than 28 voxel leads to consistently lower accuracy for both validation and test sets. Similarly, learning rates higher than 0.004 lowered the performance. One of the best performances (in terms of accuracy) may be obtained for the embodiment with a patch size of 28, freezing the first five initial layers, a mini-batch size of 40, and a learning rate of 0.006.

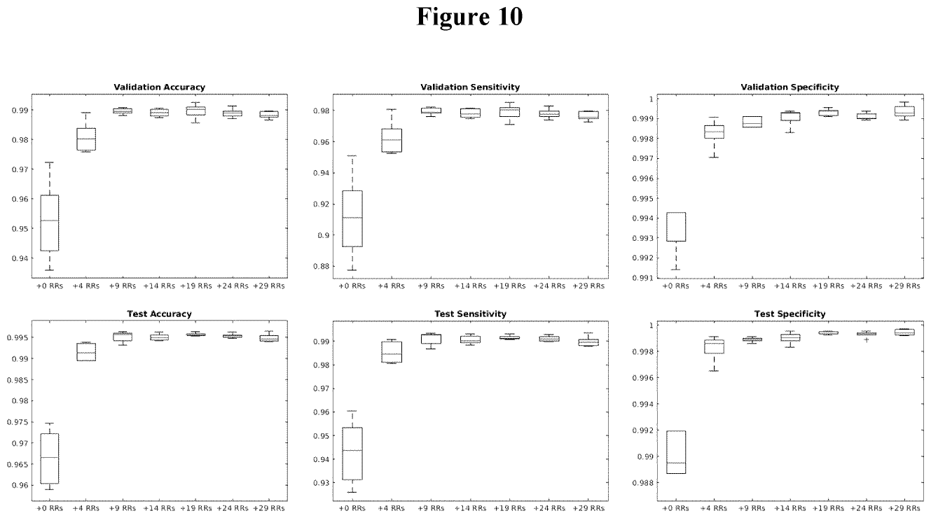

FIG. 10 shows the average performance of the model with these parameters trained with no augmentation, as well as the same model trained on original data plus data augmented with 4, 9, 14, 19, 24, and 29 random rotations (no augmentation was performed on validation and test sets). All models with data augmentation performed better than the model without any data augmentation. The better performance may be obtained using data augmented with 19 random rotations. For this network, patch-level accuracy, sensitivity, and specificity values were respectively 0.990, 0.979, and 0.999

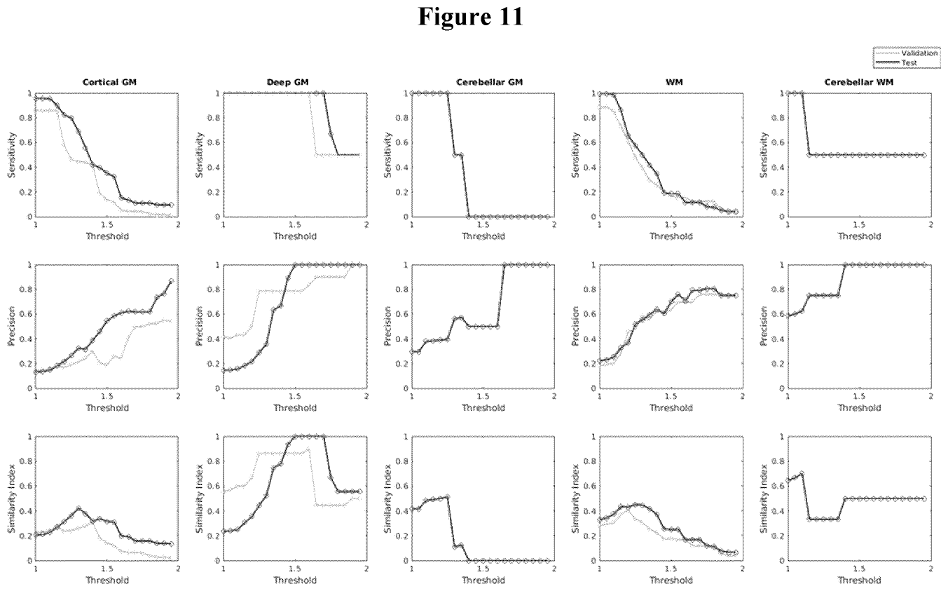

FIG. 11 shows sensitivity, precision, and similarity index values separately for cortical, deep, and cerebellar GM, and cerebral and cerebellar WM, after applying post-processing with different thresholds to the voxel-wise segmentations. The threshold values can be selected by the user based on sensitivity and precision preferences.

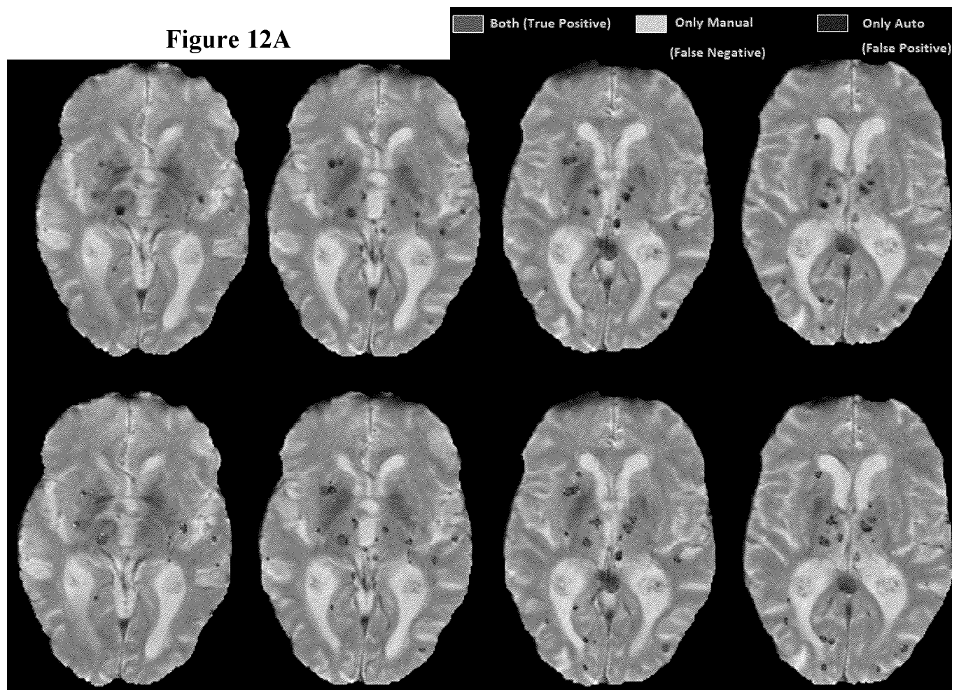

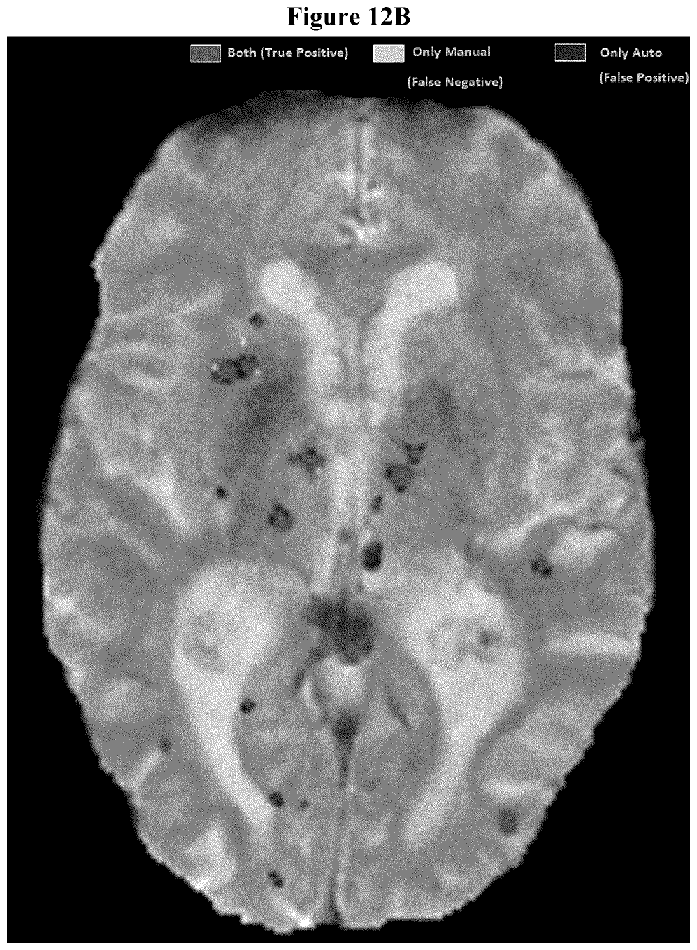

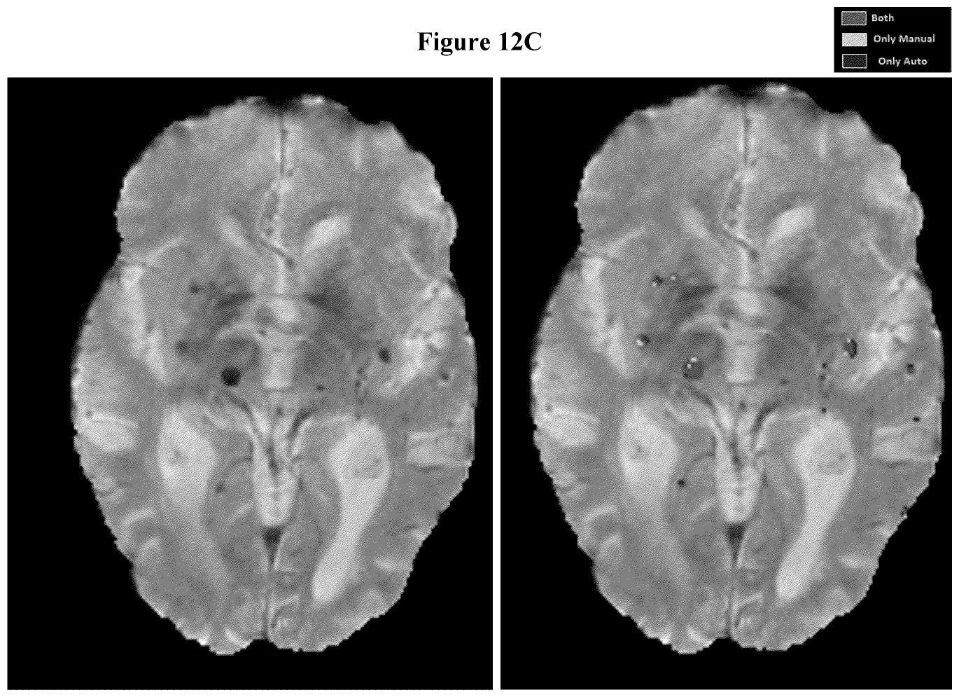

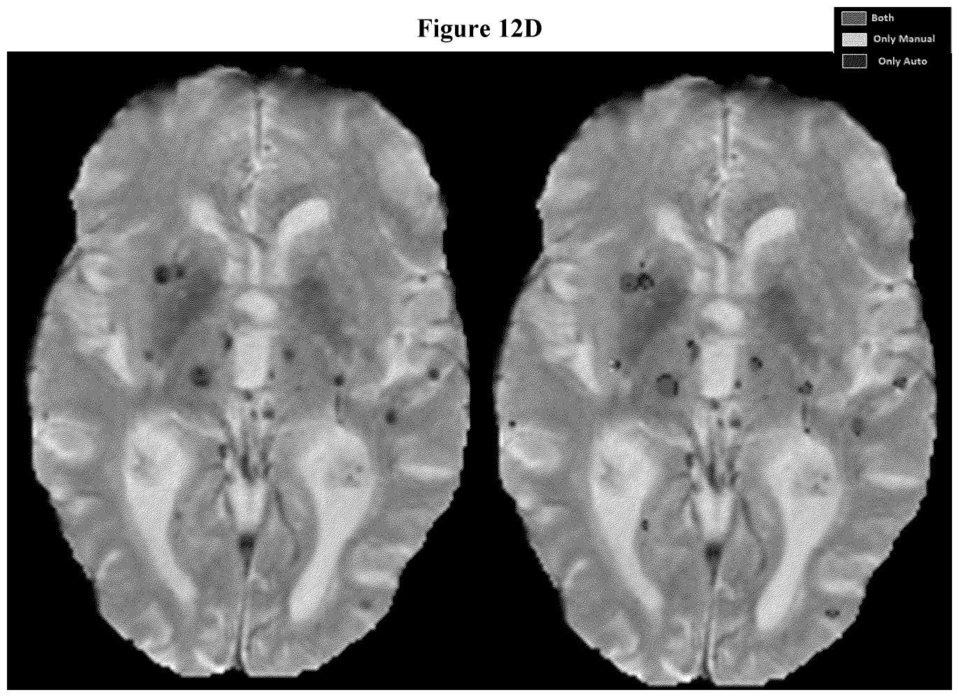

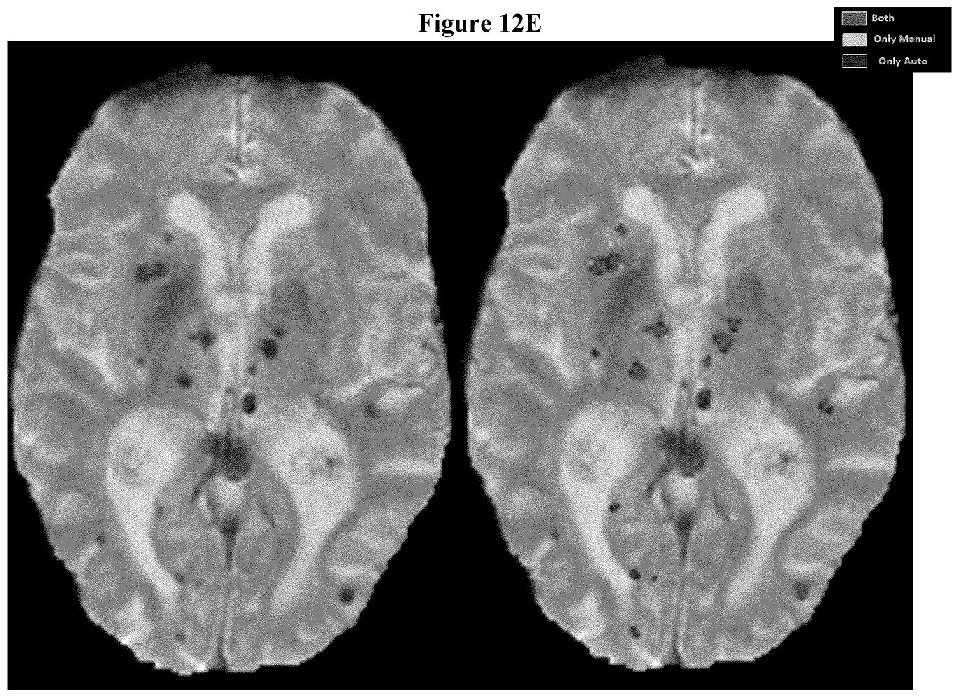

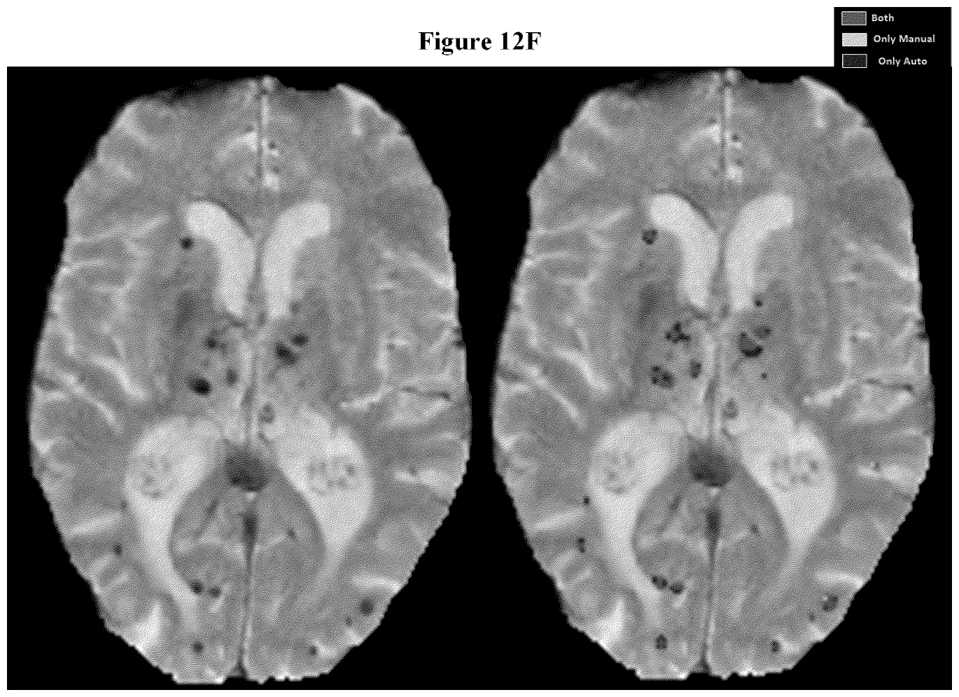

FIG. 12A shows four pairs of examples (from left to right) of automated versus manual segmentations for threshold=1.4 for the deep GM and 1.2 for the rest of the regions, with examples of true positive (indicated in red), false positive (indicated in blue), and false negative (indicated in green) classifications as illustrated in the segmented images at the bottom half of FIG. 12A (the top half present the same raw un-segmented images). Most of the disagreements are in the voxels in the border of the microbleeds, where the automated tool sometimes performs a more generous (i.e. blue voxels) or more conservative (i.e. green voxels) segmentation than the manual expert. Such differences will not affect the overall microbleed counts since both methods have essentially identified the same microbleed. FIGS. 12C to 12F are enlarged images of the four pairs (top and bottom pairs in FIG. 12A), unsegmented (left image) vs segmented (right image).

### ARIA-H Versus Microbleed

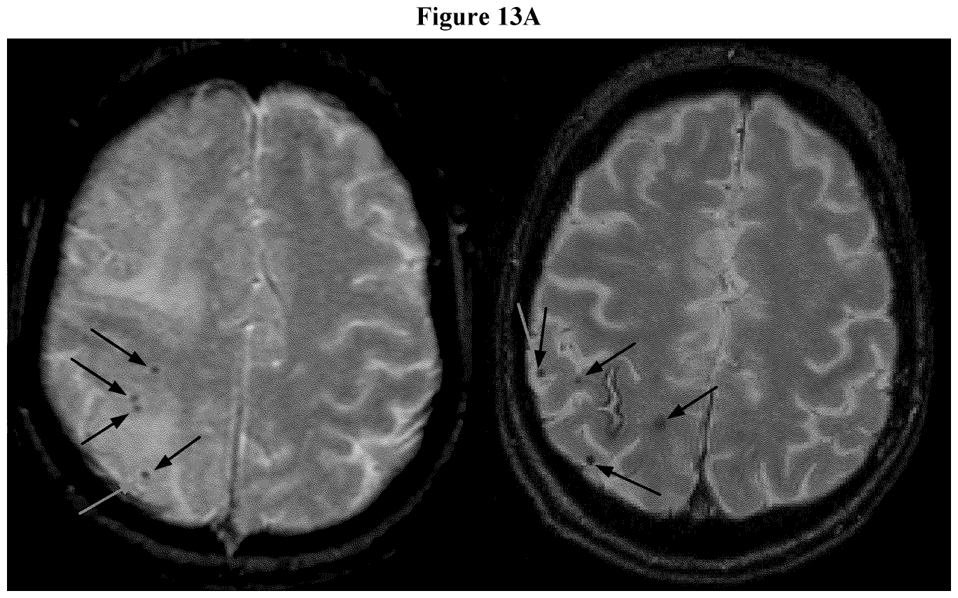

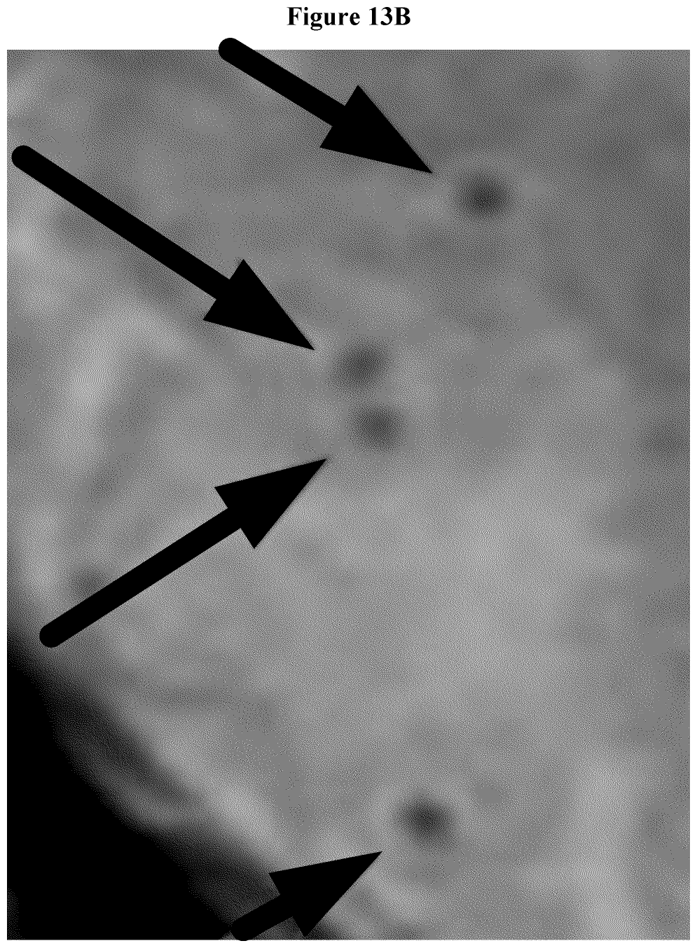

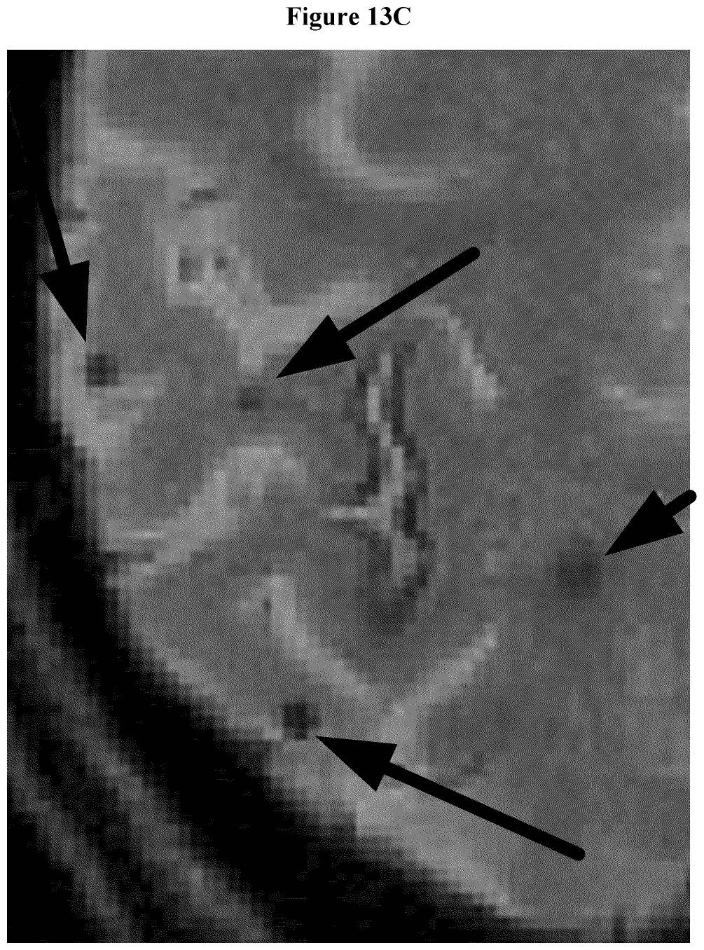

FIG. 13A present an example of ARIA-H (left image, indicated with red arrows) and microbleeds (right image, orange arrows) in the same region on T2* images. The left slice of FIG. 13A is adapted from: Arrighi, H. Michael, et al. “Amyloid-related imaging abnormalities-haemosiderin (ARIA-H) in patients with Alzheimer's disease treated with bapineuzumab: a historical, prospective secondary analysis.” Journal of Neurology, Neurosurgery & Psychiatry 87.1 (2016): 106-112. Because of their similar imaging features, applicant is of the opinion that the system and methods described herein will be able to identify, segment and quantify ARIA-H on gradient echo MRI. It will be appreciated that FIGS. 13B and 13C are zoomed-in images of a region of interest of the left and right images of FIG. 13A respectively.

### U-Net Segmentations

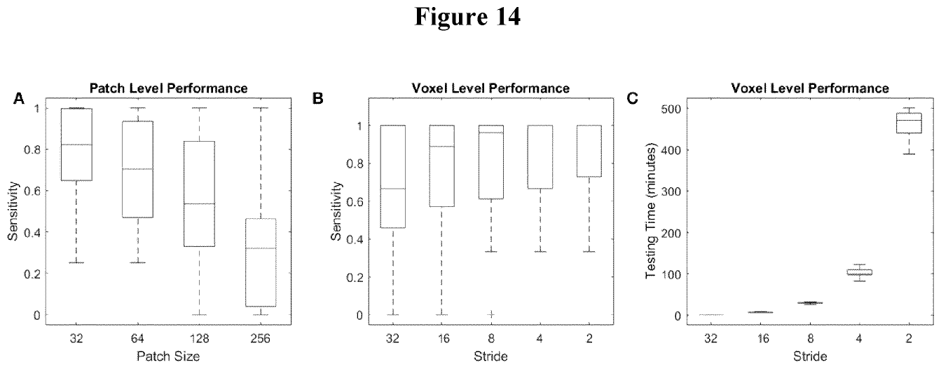

The U-Net model trained on full axial slices had similar accuracy for the CSF segmentation task to the patch-based classification model (mean Dice similarity index of 0.90±0.07), indicating that, with sufficient training data, this U-Net model can be ideal for the CSF classification task, given its low processing time (i.e., <30 s). The first graphic (A) of FIG. 14 shows the per lesion sensitivity (applied at the patch level for all patches with microbleeds) of the transfer-learned U-Net model in microbleed segmentation for different patch sizes. The full-slice model may not be able to provide accurate segmentations, missing many of the smaller microbleeds. Models trained on smaller patches had better performance, with sensitivity increasing as patch size decreased. The second graphic (B) and third graphic (C) of FIG. 14 show the performance and testing time for the most sensitive model at the patch level (i.e., patch size=32) assessed with overlapping patches, showing increased sensitivity in detecting microbleeds for smaller stride values, at the expense of an increase in testing time. Taken together, these results indicate that, when sufficient training data are not available, the less efficient patch-based models have better performance for microbleed segmentation tasks.

### DISCUSSION

Applicant presented a multi-sequence microbleed segmentation tool based on ResNet50 network and routine T1-weighted, T2-weighted, and T2* MRI. To overcome the lack of availability of a large training dataset, applicant used transfer learning to first train ResNet50 for the task of CSF segmentation, and then retrained the resulting network to perform microbleed segmentation.

Due to the unavailability of ample training data for microbleed segmentation, applicant transformed the problem at hand to a patch-based classification task, allowing us to obtain better performance at the expense of reduced efficiency and an increase in testing time. For comparison, while a U-Net model trained on complete axial slices (as opposed to smaller patches) from the same dataset may able to provide CSF segmentations with similar accuracy (mean Dice similarity index of 0.90±0.07 vs. the patch-based equivalent of 0.91±0.01), when applied in a transfer-learned model for microbleed segmentation, it may be not able to provide sufficiently accurate microbleed segmentations, missing many of the smaller microbleeds (mean sensitivity of 0.22 and mean testing time of 18 s, compared to sensitivity values >0.9 for the proposed model). Along the same line, training the U-Net segmentation model on patches improved the ability of the model to detect microbleeds (mean sensitivity values ranging from 0.64 to 0.86 with stride values from 1, ½, ¼, ⅛, and 1/16 of the patch size) at the expense of an increase in testing time (from 2 min to 7 h per case). Testing time can, however, be reduced to minutes by limiting the initial search mask (e.g., by excluding CSF voxels or areas that are hyperintense on the T2* image from the initial mask of interest since they would not include any microbleeds) or parallelizing the patch-based segmentation.

Pre-training the model on the CSF segmentation task led to a significant improvement in the segmentation results. Without performing this pre-training step, using the same model and hyper-parameters, applicant had obtained patch-level accuracy values ranging between 0.74 and 0.96 for the validation and test sets. In comparison, the final transfer learned model may be able to achieve patch-level accuracies of 0.990 and 0.996 for the validation and test sets, respectively, strongly indicative of the need for this approach.

CSF segmentation may be selected as the initial task for pre-training the model since a large number of CSF segmentations could be generated automatically without requiring manual segmentation. Furthermore, since there may not be any overlap between CSF and microbleed voxels and given that CSF voxels have a very different intensity profile than microbleeds on T2-images (CSF voxels are hyperintense in comparison with GM and WM, whereas microbleed voxels are hypointense), there would be no leakage between CSF segmentation and microbleed segmentation tasks.

The CSF classification experiments showed excellent agreement (Dice similarity index=0.91) between ResNet50 and BISON segmentations. The majority of the disagreements were in the voxels bordering the CSF and brain tissue, where ResNet50 segmented CSF slightly more generously than BISON. Given that BISON segmentations were based only on T1-weighted images, whereas ResNet50 used information from T1-weighted, T2-weighted, and T2* sequences, these voxels might actually be CSF voxels correctly classified by ResNet50 method that did not have enough contrast on T1-weighted images to be captured by BISON.

At patch level, with sensitivity and specificity values of 99.57% and 99.93%, the microbleed segmentation method outperforms most previously published results. At a per lesion level, the strategy yielded sensitivity, precision, and Dice similarity index values of 89.1%, 20.1%, and 0.28 for cortical GM, 100%, 100%, and 1 for deep GM, and 91.1%, 44.3%, and 0.58 for WM, respectively. Post-processing improved the results (increased the similarity index) for all microbleed types (by successfully removing the false positives). The improvement may be most evident for deep GM microbleeds, where most of the false positives were at the boundaries of the deep GM structures, which tend to have lower intensities compared to the neighboring tissue.

There are inherent challenges in comparing the performance of our proposed method against previously published results. Other work has been mostly based on susceptibility-weighted scans, which in general have higher sensitivity and resolution levels (usually acquired at 0.5×0.5 mm² voxels versus 1×1 mm² voxels in our case) and are better suited for microbleed detection (Dou et al., 2016; Hong et al., 2020; Roy et al., 2015; Shams et al., 2015; Van Den Heuvel et al., 2016; Wang et al., 2017; Zhang et al., 2018). Another concern in comparing results across studies regards the characteristics of the dataset used for training and validation of the results. Most previous methods have used data from populations that are much more likely to have microbleeds, such as patients with cerebral autosomal-dominant arteriopathy with subcortical infarcts and Leukoencephalopathy (CADASIL) (Hong et al., 2020; Wang et al., 2017; Zhang et al., 2018). In contrast, our dataset included non-demented aging adults and patients with neurodegenerative dementias which do not necessarily have such a high cerebrovascular disease burden. In fact, 63% of the cases in our sample had fewer than three microbleeds. Since applicant can use a participant-level assignment in training, validation, and test sets, even one false positive would reduce the per-participant precision value by 33-50% for those cases. In comparison, the training dataset used by Dou et al. included 1149 microbleeds in 20 cases (i.e. 57.45 microbleeds per case on average), in which case one false positive detection would only change the reported precision by 1.7% (Dou et al., 2016). Along the same line, applicant included very small microbleeds with volumes between 1-4 mm³ (i.e. 1 to 4 voxels) in our sample (˜35% of the total microbleed count, distributed consistently between training, validation, and test sets), whereas others might choose to not include such very small lesions which are more challenging to detect and also have lower inter and intra rater reliability (Cordonnier et al., 2007). Regardless, even considering the disadvantage of fewer microbleeds per scan inherent to our population, the proposed method compares favorably against other published results.

Generalizability to data from other scanner models and manufacturers can be another important point when developing automated segmentation tools. Automated techniques that have been developed based on data from a single scanner might not be able to reliably perform the same segmentation task when applied to data from different scanner models and with different acquisition protocols (Dadar and Duchesne, 2020). To ensure the generalizability of our results, applicant used data from seven different scanner models across three widely used scanner manufacturers (i.e. Siemens, Philips, and GE) from a number of different sites.

Due to the inherently difficult nature of the task, even in manual ratings, inter-rater and intra-rater variability in microbleed detection may generally not be very high. The per-lesion intra-rater similarity index for this exemplary dataset (based on eight randomly selected cases) was 0.82. Other studies have also reported similar results, with one study reporting intra-rater and inter-rater agreements (similarity index) of 0.93 and 0.88 respectively, while others generally report more modest inter-rater agreements ranging between 0.33-0.66 (Cordonnier et al., 2007). In a dataset of 301 patients and using T2* images for microbleed detection, Gregoire et al. reported inter-rater similarity index values of 0.68 for presence of microbleeds, where the two raters detected 375 (range: 0-35) and 507 (range: 0-49) microbleeds respectively (Gregoire et al., 2009). Given the relatively high levels of inter-rater and intra-rater variability in microbleed segmentation results, it may also be possible that some of the false positives detected by the automated method might be actual microbleed cases that were missed by the manual rater. Regarding the location of the microbleeds, Gregoire et al. reported higher levels of agreement (between the manual ratings) for microbleeds in the deep GM regions, similar to our results. This could be explained by the higher intensity contrast between the microbleeds (greater levels of hypointensity) and the background GM, which has a higher T2* signal than the WM, where the performance may usually be less robust for manual raters as well (Gregoire et al., 2009). Finally, the relatively lower performance for cortical GM microbleed segmentation can also be expected, given the close proximity to blood vessels (both in the sulci and on the surface of the brain), which show a hypointense signal that confounds with that of the microbleeds, leading to false positives and lowering the precision. However, despite the different levels of performance in the different brain areas, an automated segmentation method has the clear advantage of providing robust segmentations across different runs, essentially eliminating intra-rater variability which can be inevitable in manual segmentations.

Accurate and robust microbleed segmentation can be necessary for assessing cerebrovascular disease burden in the aging and neurodegenerative disease populations, who may show a lower number of microbleeds than other pathologies (e.g. CADASIL), making the task more challenging. Additionally, an automated tool that can reliably detect microbleeds using data from different scanner models can be highly advantageous. Our results suggest that the proposed method can provide accurate microbleed segmentations in multi-scanner data of a population with a low number of microbleed per scan, making it applicable for use in large multi-center studies.

### REFERENCES

Avants, B. B., Epstein, C. L., Grossman, M., Gee, J. C., 2008. Symmetric diffeomorphic image registration with cross-correlation: evaluating automated labeling of elderly and neurodegenerative brain. Med. Image Anal. 12, 26-41. https://doi.org/10.1016/j.media.2007.06.004
Barnes, S. R., Haacke, E. M., Ayaz, M., Boikov, A. S., Kirsch, W., Kido, D., 2011. Semiautomated detection of cerebral microbleeds in magnetic resonance images. Magn. Reson. Imaging 29, 844-852.
Bian, W., Hess, C. P., Chang, S. M., Nelson, S. J., Lupo, J. M., 2013. Computer-aided detection of radiation-induced cerebral microbleeds on susceptibility-weighted MR images. NeuroImage Clin. 2, 282-290.
Challenge, L. S. V. R., 2012. ImageNet http://www.image-net.org/challenges. LSVRC2012results Html.
Chertkow, H., Borrie, M., Whitehead, V., Black, S. E., Feldman, H. H., Gauthier, S., Hogan, D. B., Masellis, M., McGilton, K., Rockwood, K., Tierney, M. C., Andrew, M., Hsiung, G.-Y. R., Camicioli, R., Smith, E. E., Fogarty, J., Lindsay, J., Best, S., Evans, A., Das, S., Mohaddes, Z., Pilon, R., Poirier, J., Phillips, N. A., MacNamara, E., Dixon, R. A., Duchesne, S., Mackenzie, I., Rylett, R. J., 2019. The Comprehensive Assessment of Neurodegeneration and Dementia: Canadian Cohort Study. Can. J. Neurol. Sci. 46, 499-511. https://doi.org/10.1017/cjn.2019.27
Cordonnier, C., Al-Shahi Salman, R., Wardlaw, J., 2007. Spontaneous brain microbleeds: systematic review, subgroup analyses and standards for study design and reporting. Brain 130, 1988-2003.
Dadar, M., Collins, D. L., 2020. BISON: Brain tissue segmentation pipeline using T1-weighted magnetic resonance images and a random forest classifier. Magn. Reson. Med. https://doi.org/10.1002/mrm.28547
Dadar, M., Duchesne, S., 2020. Reliability assessment of tissue classification algorithms for multi-center and multi-scanner data. NeuroImage 217, 116928. https://doi.org/10.1016/j.neuroimage.2020.116928
Dadar, M., Fonov, V. S., Collins, D. L., Initiative, A. D. N., 2018. A comparison of publicly available linear MRI stereotaxic registration techniques. NeuroImage 174, 191-200.
Dadar, M., Maranzano, J., Misquitta, K., Anor, C. J., Fonov, V. S., Tartaglia, M. C., Carmichael, O. T., Decarli, C., Collins, D. L., Alzheimer's Disease Neuroimaging Initiative, 2017a. Performance comparison of 10 different classification techniques in segmenting white matter hyperintensities in aging. NeuroImage 157, 233-249. https://doi.org/10.1016/j.neuroimage.2017.06.009
Dadar, M., Narayanan, S., Arnod, D. L., Collins, D. L., Maranzano, J., 2020. Conversion of Diffusely Abnormal White Matter to Focal Lesions is Linked to Progression in Secondary Progressive Multiple Sclerosis. Mult. Scler. J. 832345.
Dadar, M., Pascoal, T., Manitsirikul, S., Misquitta, K., Tartaglia, C., Brietner, J., Rosa-Neto, P., Carmichael, O., DeCarli, C., Collins, D. L., 2017b. Validation of a Regression Technique for Segmentation of White Matter Hyperintensities in Alzheimer's Disease. IEEE Trans. Med. Imaging.
Dadar, M., Potvin, O., Camicioli, R., Duchesne, S., Initiative, A. D. N., 2021. Beware of white matter hyperintensities causing systematic errors in FreeSurfer gray matter segmentations! Hum. Brain Mapp.
Dice, L. R., 1945. Measures of the amount of ecologic association between species. Ecology 26, 297-302.
Dou, Q., Chen, H., Yu, L., Zhao, L., Qin, J., Wang, D., Mok, V. C., Shi, L., Heng, P.-A., 2016. Automatic detection of cerebral microbleeds from MR images via 3D convolutional neural networks. IEEE Trans. Med. Imaging 35, 1182-1195.
Duchesne, S., Chouinard, I., Potvin, O., Fonov, V. S., Khademi, A., Bartha, R., Bellec, P., Collins, D. L., Descoteaux, M., Hoge, R., McCreary, C. R., Ramirez, J., Scott, C. J. M., Smith, E. E., Strother, S. C., Black, S. E., 2019. The Canadian Dementia Imaging Protocol: Harmonizing National Cohorts. J. Magn. Reson. Imaging 49, 456-465. https://doi.org/10.1002/jmri.26197
Eskildsen, S. F., Coupé, P., Fonov, V., Manjón, J. V., Leung, K. K., Guizard, N., Wassef, S. N., Østergaard, L. R., Collins, D. L., Initiative, A. D. N., others, 2012. BEaST: brain extraction based on nonlocal segmentation technique. NeuroImage 59, 2362-2373.
Fazlollahi, A., Meriaudeau, F., Giancardo, L., Villemagne, V. L., Rowe, C. C., Yates, P., Salvado, O., Bourgeat, P., Group, A. R., 2015. Computer-aided detection of cerebral microbleeds in susceptibility-weighted imaging. Comput. Med. Imaging Graph. 46, 269-276.
Fischl, B., 2012. FreeSurfer. Neuroimage 62, 774-781.
Greenberg, S. M., Vernooij, M. W., Cordonnier, C., Viswanathan, A., Al-Shahi Salman, R., Warach, S., Launer, L. J., Van Buchem, M. A., Breteler, M. M., 2009. Cerebral microbleeds: a guide to detection and interpretation. Lancet Neurol. 8, 165-174. https://doi.org/10.1016/S1474-4422 (09) 70013-4
Gregoire, S. M., Chaudhary, U. J., Brown, M. M., Yousry, T. A., Kallis, C., Jäger, H. R., Werring, D. J., 2009. The Microbleed Anatomical Rating Scale (MARS): reliability of a tool to map brain microbleeds. Neurology 73, 1759-1766.
He, K., Zhang, X., Ren, S., Sun, J., 2016. Deep residual learning for image recognition, in: Proceedings of the IEEE Conference on Computer Vision and Pattern Recognition. pp. 770-778.
Hong, J., Cheng, H., Zhang, Y.-D., Liu, J., 2019. Detecting cerebral microbleeds with transfer learning. Mach. Vis. Appl. 30, 1123-1133.
Hong, J., Wang, S.-H., Cheng, H., Liu, J., 2020. Classification of cerebral microbleeds based on fully-optimized convolutional neural network. Multimed. Tools Appl. 79, 15151-15169.
Krizhevsky, A., Sutskever, I., Hinton, G. E., 2017. Imagenet classification with deep convolutional neural networks. Commun. ACM 60, 84-90.
Kuijf, H. J., Brundel, M., Bresser, J. de, Veluw, S. J. van, Heringa, S. M., Viergever, M. A., Biessels, G. J., Vincken, K. L., 2013. Semi-Automated Detection of Cerebral Microbleeds on 3.0 T MR Images. PLOS ONE 8, e66610. https://doi.org/10.1371/journal.pone.0066610
Kuijf, H. J., de Bresser, J., Geerlings, M. I., Conijn, M. M., Viergever, M. A., Biessels, G. J., Vincken, K. L., 2012. Efficient detection of cerebral microbleeds on 7.0 T MR images using the radial symmetry transform. Neuroimage 59, 2266-2273.
Lu, S., Lu, Z., Hou, X., Cheng, H., Wang, S., 2017. Detection of cerebral microbleeding based on deep convolutional neural network, in: 2017 14th International Computer Conference on Wavelet Active Media Technology and Information Processing (ICCWAMTIP). IEEE, pp. 93-96.
Manera, A. L., Dadar, M., Fonov, V., Collins, D. L., 2020. CerebrA, registration and manual label correction of Mindboggle-101 atlas for MNI-ICBM152 template. Sci. Data 7, 1-9.
Maranzano, J., Dadar, M., Arnold, D. L., Collins, D. L., Narayanan, S., 2020. Automated Separation of Diffusely Abnormal White Matter from Focal White Matter Lesions on MRI in Multiple Sclerosis. NeuroImage 727818.
Mateos-Pérez, J. M., Dadar, M., Lacalle-Aurioles, M., Iturria-Medina, Y., Zeighami, Y., Evans, A. C., 2018. Structural neuroimaging as clinical predictor: A review of machine learning applications. NeuroImage Clin. https://doi.org/10.1016/j.nicl.2018.08.019
Morrison, M. A., Payabvash, S., Chen, Y., Avadiappan, S., Shah, M., Zou, X., Hess, C. P., Lupo, J. M., 2018. A user-guided tool for semi-automated cerebral microbleed detection and volume segmentation: Evaluating vascular injury and data labelling for machine learning. NeuroImage Clin. 20, 498-505. https://doi.org/10.1016/j.nicl.2018.08.002
Pieruccini-Faria, F., Black, S. E., Masellis, M., Smith, E. E., Almeida, Q. J., Li, K. Z. H., Bherer, L., Camicioli, R., Montero-Odasso, M., 2021. Gait variability across neurodegenerative and cognitive disorders: Results from the Canadian Consortium of Neurodegeneration in Aging (CCNA) and the Gait and Brain Study. Alzheimers Dement. n/a. https://doi.org/10.1002/alz.12298
Roob, G., Schmidt, R., Kapeller, P., Lechner, A., Hartung, H.-P., Fazekas, F., 1999. MRI evidence of past cerebral microbleeds in a healthy elderly population. Neurology 52, 991-991.
Roy, S., Jog, A., Magrath, E., Butman, J. A., Pham, D. L., 2015. Cerebral microbleed segmentation from susceptibility weighted images, in: Medical Imaging 2015: Image Processing. International Society for Optics and Photonics, p. 94131E.
Shams, S., Martola, J., Cavallin, L., Granberg, T., Shams, M., Aspelin, P., Wahlund, L. O., Kristoffersen-Wiberg, M., 2015. SWI or T2*: which MRI sequence to use in the detection of cerebral microbleeds? The Karolinska Imaging Dementia Study. Am. J. Neuroradiol. 36, 1089-1095.
Shoamanesh, A., Kwok, C. S., Benavente, O., 2011. Cerebral microbleeds: histopathological correlation of neuroimaging. Cerebrovasc. Dis. 32, 528-534.
Simonyan, K., Zisserman, A., 2014. Very deep convolutional networks for large-scale image recognition. ArXiv Prepr. ArXiv14091556.
Sled, J. G., Zijdenbos, A. P., Evans, A. C., 1998. A nonparametric method for automatic correction of intensity nonuniformity in MRI data. Med. Imaging IEEE Trans. On 17, 87-97.
Sveinbjornsdottir, S., Sigurdsson, S., Aspelund, T., Kjartansson, O., Eiriksdottir, G., Valtysdottir, B., Lopez, O. L., Van Buchem, M. A., Jonsson, P. V., Gudnason, V., 2008. Cerebral microbleeds in the population based AGES-Reykjavik study: prevalence and location. J. Neurol. Neurosurg. Psychiatry 79, 1002-1006.
Szegedy, C., Liu, W., Jia, Y., Sermanet, P., Reed, S., Anguelov, D., Erhan, D., Vanhoucke, V., Rabinovich, A., 2015. Going deeper with convolutions, in: Proceedings of the IEEE Conference on Computer Vision and Pattern Recognition. pp. 1-9.
Van Den Heuvel, T. L. A., Van Der Eerden, A. W., Manniesing, R., Ghafoorian, M., Tan, T., Andriessen, T., Vyvere, T. V., Van den Hauwe, L., Ter Haar Romeny, B. M., Goraj, B. M., 2016. Automated detection of cerebral microbleeds in patients with traumatic brain injury. NeuroImage Clin. 12, 241-251.
Vernooij, M. W., van der Lugt, A., Ikram, M. A., Wielopolski, P. A., Niessen, W. J., Hofman, A., Krestin, G. P., Breteler, M. M. B., 2008. Prevalence and risk factors of cerebral microbleeds: the Rotterdam Scan Study. Neurology 70, 1208-1214.
Wang, S., Jiang, Y., Hou, X., Cheng, H., Du, S., 2017. Cerebral Micro-Bleed Detection Based on the Convolution Neural Network With Rank Based Average Pooling. IEEE Access 5, 16576-16583. https://doi.org/10.1109/ACCESS.2017.2736558
Wardlaw, J. M., Smith, E. E., Biessels, G. J., Cordonnier, C., Fazekas, F., Frayne, R., Lindley, R. I., O'Brien, J. T., Barkhof, F., Benavente, O. R., 2013. STandards for ReportIng Vascular changes on nEuroimaging (STRIVE v1). Neuroimaging standards for research into small vessel disease and its contribution to ageing and neurodegeneration. Lancet Neurol 12, 822-838.
Werring, D. J., Frazer, D. W., Coward, L. J., Losseff, N. A., Watt, H., Cipolotti, L., Brown, M. M., Jäger, H. R., 2004. Cognitive dysfunction in patients with cerebral microbleeds on T2*-weighted gradient-echo MRI. Brain 127, 2265-2275.
Zhang, Y.-D., Hou, X.-X., Chen, Y., Chen, H., Yang, M., Yang, J., Wang, S.-H., 2018. Voxelwise detection of cerebral microbleed in CADASIL patients by leaky rectified linear unit and early stopping. Multimed. Tools Appl. 77, 21825-21845. https://doi.org/10.1007/s11042-017-4383-9
Nakata-Kudo Y, Mizuno T, Yamada K, et al. Microbleeds in Alzheimer disease are more related to cerebral amyloid angiopathy than cerebrovascular disease. Dement Geriatr Cogn Disord 2006; 22:8-14. 10.1159/000092958 [PubMed] [CrossRef] [Google Scholar]
Sperling R A, Jack C R Jr, Black S E, et al. Amyloid-related imaging abnormalities in amyloid-modifying therapeutic trials: recommendations from the Alzheimer's Association Research Roundtable Workgroup. Alzheimer Dement 2011; 7:367-85. 10.1016/j.jalz.2011.05.2351 [PMC free article] [PubMed] [CrossRef] [Google Scholar]
Zago W, Schroeter S, Guido T, et al. Vascular alterations in PDAPP mice after anti-A immunotherapy: implications for amyloid-related imaging abnormalities. Alzheimer Dement 2013; 9: S105-15. 10.1016/j.jalz.2012.11.010 [PubMed] [CrossRef] [Google Scholar]
H Michael Arrighi, 1 Jerome Barakos, 2,3 Frederik Barkhof, 4,5 Donatella Tampieri, 6 Clifford Jack, Jr, 7 Denis Melançon, 7Kristen Morris, 8,9 Nzeera Ketter, 1 Enchi Liu, 1 and H Robert Brashear1, Amyloid-related imaging abnormalities-haemosiderin (ARIA-H) in patients with Alzheimer's disease treated with bapineuzumab: a historical, prospective secondary analysis, J Neurol Neurosurg Psychiatry. 2016 January; 87 (1): 106-112.
Nzeera Ketter 1, H Robert Brashear 1, Jennifer Bogert 2, Jianing Di 1, Yves Miaux 3, Achim Gass 3, Derk D Purcell 3, Frederik Barkhof 4, H Michael Arrighi 1. Central Review of Amyloid-Related Imaging Abnormalities in Two Phase III Clinical Trials of Bapineuzumab in Mild-To-Moderate Alzheimer's Disease Patients.
Lawren VandeVrede 1, Daniel M Gibbs 1, Mary Koestler 1, Renaud La Joie 1, Peter A Ljubenkov 1, Karine Provost 1, David Soleimani-Meigooni 1, Amelia Strom 1, Elena Tsoy 1, Gil D Rabinovici 1 2, Adam L Boxer 1. Symptomatic amyloid-related imaging abnormalities in an APOE ε4/ε4 patient treated with aducanumab. J Alzheimers Dis. 2017; 57 (2): 557-573. 2020 Oct. 9; 12 (1): e12101.

1. A method of processing a brain image to determine presence of microbleeds, the method comprising:
providing a classifier trained to recognize microbleed voxels;
receiving a brain image of a subject; and
identifying microbleed voxels in said brain image using said classifier;
wherein false positives in said identified microbleed voxels are reduced by at least one of:
removing identified microbleed voxels based on a comparison between an image characteristic of said identified microbleed voxels and of one or more surrounding non-microbleed voxels; and
said classifier being first trained on a relevant image characteristic for distinguishing cerebral spinal fluid from gray matter/white matter using a first training set of brain images and being further trained to distinguish microbleed voxels from other brain tissue voxels using a second set of microbleed brain images segmented by one or more experts.

2. The method of claim 1, wherein false positives in said identified microbleed voxels are reduced by removing identified microbleed voxels based on a comparison between an image characteristic of said identified microbleed voxels and of one or more surrounding non-microbleed voxels.

3. The method of claim 2, wherein said brain image is preprocessed for intensity non-uniformity correction and/or linear intensity standardization.

4. The method of claim 2, wherein said providing a classifier comprises:
training said classifier on a relevant image characteristic for distinguishing cerebral spinal fluid from gray matter/white matter using a first training set of brain images;
further training said classifier to distinguish microbleed voxels from other brain tissue voxels using a second set of microbleed brain images segmented by one or more experts.

5. (canceled)

6. The method of claim 4, wherein said training said classifier on a relevant image characteristic using a first training set of brain images comprises using patches of voxels taken from training images

7. The method of claim 4, wherein said further training comprises using patches of voxels from said second set of microbleed brain images in different rotational positions.

8. The method of claim 4, wherein said classifier trained to recognize microbleed voxels has an accuracy greater than 95%.

9. (canceled)

10. The method of claim 1, wherein said classifier being first trained on a relevant image characteristic for distinguishing cerebral spinal fluid from gray matter/white matter using a first training set of brain images and being further trained to distinguish microbleed voxels from other brain tissue voxels using a second set of microbleed brain images segmented by one or more experts.

11. The method of claim 10, wherein said image characteristic is an intensity value.

12. The method of claim 10, wherein said brain image is preprocessed for intensity non-uniformity correction and/or linear intensity standardization.

13. The method of claim 10, wherein said training said classifier on a relevant image characteristic using a first training set of brain images comprises using patches of voxels taken from training images.

14. The method of claim 13, wherein said further training comprises using patches of voxels from said second set of microbleed brain images in different rotational positions.

15. The method of claim 10, wherein said classifier trained to recognize microbleed voxels has an accuracy greater than 95%.

16. A method of treating a patient using amyloid-modifying therapy, the method comprising:
a. obtaining a brain image of said patient;
b. processing said brain image of said patient using the method of claim 1; and
c. administering said therapy to a based on said identifying microbleed voxels related to amyloid-related microbleeds.

17. The method of claim 16, wherein steps (a) through (c) are repeated over time and said modifying comprises comparing changes in identified microbleed voxels over time.

18. An apparatus for processing a brain image to determine presence of microbleeds, the apparatus comprising:
a processor and non-volatile memory storing processor instructions that when executed by the processor perform:
providing a classifier trained to recognize microbleed voxels;
receiving a brain image of a subject; and
identifying microbleed voxels in said brain image using said classifier;
wherein false positives in said identified microbleed voxels are reduced by at least one of:
removing identified microbleed voxels based on a comparison between an image characteristic of said identified microbleed voxels and of one or more surrounding non-microbleed voxels; and
said classifier being first trained on a relevant image characteristic for distinguishing cerebral spinal fluid from gray matter/white matter using a first training set of brain images and being further trained to distinguish microbleed voxels from other brain tissue voxels using a second set of microbleed brain images segmented by one or more experts.
